(19) United States
(12) Patent Application Publication
(10) Pub. No.: US 2011/0059018 A1
(43) Pub. Date: Mar. 10, 2011

(54) COVALENTLY BINDING IMAGING PROBES

(75) Inventors: Karl-Ulrich WENDT, Frankfurt am Main (DE); Maik KINDERMANN, Basel (CH); Catherine MINIEJEW, Frankfurt am Main (DE); Anja GLOBISCH, Dortmund (DE)

(73) Assignee: SANOFI-AVENTIS, Paris (FR)

(21) Appl. No.: 12/860,544

(22) Filed: Aug. 20, 2010

Related U.S. Application Data
(63) Continuation of application No. PCT/EP2009/000815, filed on Feb. 6, 2009.

(30) Foreign Application Priority Data
Feb. 21, 2008 (EP) 08290167.9

(51) Int. Cl. A61K 49/00; C07D 295/215; C07D 295/155; C07D 487/04; C07D 413/12; C07D 495/04; C07D 311/82; C07D 403/06; C12Q 1/02; G01N 21/00

(52) U.S. Cl. 424/9.1; 544/159; 544/400; 540/500; 544/150; 544/139; 549/394; 548/455; 548/303.7; 435/29; 436/164

(57) ABSTRACT

The present invention relates to molecular probes of the formula (I)

L1-R1-L-A-X (I)

as defined herein that allow for the observation of the catalytic activity of a selected caspase, cathepsin, MMP and carboxypeptidase in in vitro assays, in cells or in multicellular organisms, a method for their preparation and the use thereof.

### FIELD OF THE INVENTION

The present invention relates to molecular probes (inhibitors) that allow for the observation of the catalytic activity of individual proteolytic enzymes or groups of proteolytic enzymes in in vitro assays, in cells or in multicellular organisms. The invention furthermore relates to methods for the synthesis and the design of such probes (inhibitors).

### BACKGROUND OF THE INVENTION

Proteolytic enzymes (proteases) cleave or degrade other enzymes or peptides in- and outside of the living cell. Proteases are involved in a multitude of vital processes, many of which are critical in cellular signalling and tissue homeostasis. Aberrant or enhanced activity of proteases is associated with a variety of diseases including cancer, osteoarthritis, arteriosclerosis, inflammation and many others (M. J. Evans, B. F. Cravatt, Chem. Rev. 2006, 106, 3279-3301). Since proteolytic activity has to remain under stringent control in living systems many proteases are expressed as inactive precursor proteins (zymogens) which are activated by controlled proteolytic cleavage. Additional control of proteolytic activity results from endogenous inhibitors that bind to and thereby inactivate catalytically active form of the enzyme. In view of this stringent regulation the investigation of protease function in cellular or physiological events requires the monitoring of protease activity rather than the monitoring of protease expression alone. Consequently, a variety of chemical probes have been proposed in the literature. Commonly applied protease probes generate a detectable signal either (i) through enzymatic cleavage of a peptide bond leading the spatial separation of a fluorophore from a fluorescence quencher or (ii) by covalent attachment of a mechanism based inhibitor to the protease of interest. The localization and quantitative investigation of the activity and inhibition of a specific protease or a group of proteases (e.g. in cell-based assays or whole-animal imaging experiments) require the development of imaging probes that (i) reach the physiologically relevant locus of protease action (e.g. the cytosol of a cell or a specific organ in whole animal imaging) and (ii) are selective for the desired protease or a group of proteases. The generation of protease selective probes has imposed a considerable challenge for the field. The present invention relates (i) to novel highly selective probes for cysteine proteases preferably from the cathepsin or caspase subfamilies, and for metalloproteases preferably from matrix metalloprotease (MMP) or carboxypeptidase subfamilies (ii) to the application of these probes in vitro assays, in cells or in multicellular organisms (e.g. by the means of molecular imaging) and (iii) to methods for the synthesis and the design of such probes.

Within recent years several molecular imaging technologies (optical and non-optical) have become more and more important for the visualization of specific molecular targets and pathways. The generation of probes that are selective for individual proteases and exhibit the ability to reach the locus of protease action in vivo has rarely been achieved with conventional approaches. Medicinal chemists in the pharmaceutical industry face related challenges in the development of drugs with appropriate pharmacokinetic properties and appropriate specificity for a given target. In our invention we have devised about new chemical scaffolds towards selective probes for cysteine proteases from the cathepsin or caspase subfamilies, and for metalloproteases preferably from matrix metalloprotease (MMP) or carboxypeptidase subfamilies.

Cysteine proteases are characterized by a cysteine residue in the active site which serves as a nucleophile during catalysis. The catalytic cysteine is commonly hydrogen bonded with appropriate neighboring residues, so that a thiolate ion can be formed. When a substrate is recognized by the protease, the scissile peptide bond is placed in proximity to the catalytic cysteine, which attacks the carbonyl carbon forming an oxoanion intermediate. The amide bond is then cleaved liberating the C-terminal peptide as an amine. The N-terminal portion of the scissile peptide remains in the covalent acyl-enzyme intermediate, which is subsequently cleaved by water, resulting in regeneration of the enzyme. The N-terminal cleavage product of the substrate is liberated as a carboxylic acid.

The human genome encodes 11 papaine-like cathepsins (human clan CA proteases or the cysteine cathepsins: B, C, F, H, K, L, O, S, V, W, X) which are implicated with various functions including general protein degradation in lysosomes (housekeeping function), processing of antigens, processing of granular proteases, and matrix collagen degradations. Malfunction of cysteine cathepsins have been associated with a number of pathological events such as osteoarthritis, cancer biology (angiogenesis and tumorigenesis), neurological disorders (e.g. pain) and osteoporosis (Y. Yasuda et al. Adv. Drug Delivery Rev. 2005, 57, 973-993) and consequently some of the cysteine cathepsins have been validated as relevant drug targets for therapies over recent years (Turk, V.; Turk, B.; Turk, D. Embo J, 2001, 20, 4629-4633).

For example, Cathepsin K and S are implicated in bone and cartilage degradation and are related to osteoporosis and arthritis.

Furthermore, Cathepsin K is predominantly found in osteoclasts and was shown to bee crucial for normal bone remodeling (bone resorption). A deficiency of Cathepsin K activity results in a bone sclerosis disorder (pycnodysostosis), whereas over expression in cathepsin K accelerated the turnover of bone material as it is indicative for osteoporosis. Cathepsin K also shows potent collagenase activity, cleaving triple helical collagens in their helical domains. In Osteoarthritis the cartilage matrix is undergoing massive erosion including the degradation of type II collagen (Y. Yasuda et al. Adv. Drug Delivery Rev. 2005, 57, 973-993). Thus, inhibition of Cathepsin B and K, for example, is a useful method for the treatment of degenerative joint diseases such as, for example, osteoarthritis. Cathepsin K inhibition, for example, leads to inhibition of bone. Cathepsin S plays a major role to initiate a MHC class II related immune response towards an antigen. Being the main invariant cain-processing protease in dendritic cells, Cathepsin S appears as attractive drug target in immune related diseases. Furthermore Cathepsin S might be also important for extracellular matrix degradation and shows significant elastase and proteoglycan-degrading activity. Cathepsin S is therefore implicated in disorders involving excessive elastolysis, such as chronic obstructive pulmonary disease (e.g. emphysema), bronchiolitis, excessive airway elastolysis in asthma and bronchitis, pneumonities and cardiovascular disease such as plaque rupture and atheroma.

Cathepsin L appears to be involved in epidermal homeostasis, regulation of the hair cycle and also MHC class II-mediated antigen presentation.

Cathepsin B is associated with pathological trypsin activation in the early stage of pancreatitis and contributes to TNF-alpha induced hepatocyte apoptosis.

Caspases are a family of cysteinyl aspartate-specific proteases. The human genome encodes 11 caspases. Eight of them (caspase-2,3,6,7,8,9,10 and 14) function in apoptosis or programmed cell death. They process through a highly regulated signalling cascade. In a hierarchical order, some initiator caspases (caspase-2,8,9 and 10) cleave and activate effector caspases (caspase-3,6 and 7). These caspases are involved in cancers, autoimmune diseases, degenerative disorders and strokes. Three other Caspases (caspase-1, 4 and 5) serve a distinct function: inflammation mediated by activation of a subset of inflammatory cytokines.

Caspase-1 or interleukin-1β-converting enzyme (ICE) is primarily found in monocytic cells. This protease is responsible for the production of the pro-inflammatory cytokines interleukin-1-beta and interleukine-18. Inhibition of caspase-1 has been shown to be beneficial in models of human inflammation disease, including rheumatoid arthritis, osteoarthritis, inflammatory bowel disease and asthma.

Caspase-3 is responsible for proteolitic cleavage of a variety of fundamental proteins including cytoskeletal proteins, kinases and DNA-repair enzymes. It is a critical mediator of apoptosis in neurons. Inhibition of caspase-3 have shown efficacy in models such as stroke, traumatic brain spinal cord injury, hypoxic brain damage, cardiac ischemia and reperfusion injury.

Caspase-8 is an apoptosis initiator caspase, downstream of TNF super-family death receptors. Its substrates include apoptosis-related effector caspases and pro-apoptotic Bcl-2 family members. Resistance to apoptosis in cancer has been linked to low expression levels of caspase-8 and inhibition of caspase-8 increases resistance to apoptosis-inducing stressors such as chemotherapy and radiation. Thus caspase-8 is an attractive target for therapy of tumours and metastatic lesions. Knockout studies reveal as well several other potential roles for caspases-8 which are independent of apoptosis. For example, caspase-8 knockouts exhibit deficiencies in leukocyte differentiation, proliferation and immune response.

Metalloproteases constitute a family of proteases which bind at least one metal ion in their active site.

Matrix metalloproteinases (MMPs) are a large family of calcium-dependent zinc-containing endopeptidases, which are responsible for the tissue remodeling and degradation of the extracellular matrix (ECM), including collagens, elastins, gelatin, matrix glycoproteins, and proteoglycan. MMPs are usually minimally expressed in normal physiological conditions and thus homeostasis is maintained. However, MMPs are regulated by hormones, growth factors, and cytokines, and are involved in ovarian functions. Endogenous MMP inhibitors (MMPIs) and tissue inhibitors of MMPs (TIMPs) strictly control these enzymes. Over-expression of MMPs results in an imbalance between the activity of MMPs and TIMPs that can lead to a variety of pathological disorders including the destruction of cartilage and bone in rheumatoid arthritis and osteoarthritis, tumours growth and metastasis in both human and animal cancers (R. Cowling et al. J. Med. Cem. 2003, 46, 2361; K. U. Wendt, C. K. Engel et al. Chem. Biol. 2005, 12, 181; W. J. Welsh et al. J. Med. Chem. 2001, 44, 3849; D. Barone et al. J. Med. Chem. 2004, 47, 6255). To date at least 26 human MMPs are known. On the basis of their specificity, these MMPs are classified into collagenases, gelatinases, stromelysins, and matrilysins. The majority of the MMPs are divided into four main groups that include collagenases (MMP-1, -8, -13), gelatinases (MMP-2, -9), stromelysins (MMP-3, -10, -1) and membrane-type MMPs (MMP-14, -15, -16, -17), while matrilysin (MMP-7) and metalloelastase (MMP-12) are included separately as members of the metalloproteinase family (for review see: C. Hansch et al. Bioorg. Med. Chem. 2007, 15, 2223-2268).

The reaction mechanism for the proteolysis by MMPs has been delineated on the basis of structural information (R. L. Stein et al. Biochemistry 1992, 31, 10757; S. R. Jordan Biochemistry 1994, 33, 8207). It is proposed that the scissile amide carbonyl coordinates to the active-site zinc(II) ion. This carbonyl is attacked by a water molecule, which is both hydrogen bonded to a conserved glutamic acid and coordinated to the zinc(II) ion.

Carboxypeptidases are exopeptidases that catalyze the hydrolysis of peptide bound at the C-terminus of peptides and proteins. They can be subdivided based on their involvement in specific physiological processes. Pancreatic carboxypeptidases function as digestive enzyme whereas regulatory carboxypeptidases exert their action in various physiological processes, mainly in non-digestive tissues and fluids.

Carboxypeptidase U or thrombin activable fibrinolysis inhibitor (TAFI) is found in blood as zymogen and is activated by the thrombin. It protects the fibrin clot against lysis. It is involved in bleeding and thrombotic disorders as well as in blood pressure regulation, inflammation or wound healing. Inhibitors of TAFI are for example important for the treatment of patient with a hypercoagulant status or for the prevention of deep vein thrombosis.

For proteolytic enzymes, it is their activity, rather than mere expression level, that dictates their functional role in cell physiology and pathology. Accordingly, molecules that inhibit the activity of proteases are useful as therapeutic agents in the treatment of diseases and the development of specific imaging biomarkers that visualize the proteolytic activity as well as their inhibition through drug candidates may accelerate target validation, drug development and even clinical trials (H. Pien, A. J. Fischman, J. H. Thrall, A. G. Sorensen, Drug Discovery Today, 2005, 10, 259-266). Using imaging reagents, a specific protein or protein family can be readily monitored in complex protein mixtures, intact cells, and even in vivo. Furthermore, enzyme class specific probes can be used to develop screens for small molecule inhibitors that can be used for functional studies (D. A. Jeffery, M. Bogyo Curr. Opp. Biotech. 2003, 14, 87-95).

So far, imaging probes incorporating a peptide substrate have been developed to monitor and label cathepsin B and L in cell based assays (G. Blum et al. Nat. Chem. Biol, 2005, 1, 203-209), several cathepsins (R. Weissleder et al. Nat. Biotech. 1999, 17, 375-378) and matrix metalloproteinases in tumours tissue (C. Bremer et al. Nat. Med. 2001, 7, 743-748). Imaging probes incorporating a peptide substrate have been developed as well to monitor and label in cell based assays caspase-1 (W. Nishii et al., FEBS Letters 2002, 518, 149-153), caspase-3 (S. Mizukami et al., FEBS Letters 1999, 453, 356-360, A. Berger, M. Bogyo et al. Mol. Cell, 2006, 23, 509-521) or caspases-8 (A. Berger, M. Bogyo et al. Mol. Cell, 2006, 23, 509-521). Furthermore a near-infrared fluorescent probe has been reported to detect caspase-1 activity in living animals (S. Messerli et al., Neoplasia 2004, 6, 95-105).

The enzymatic mechanism used by some proteases has been well studied and is highly conserved. From the investigation and screening data of cleavable peptides, electrophilic substrate analogs have been developed that only react in the context of this conserved active site. The electrophilic center in such probes is usually part of a so called “warhead”, a molecular entity that is optimized in its electrophilic character and its geometric placement to fit perfectly into the active site of a protease, where it reacts with the catalytic residue. A wide variety of such electrophilic substrates have been described as mechanism based protease inhibitors including for example but not exclusively: diazomethyl ketones, fluoromethyl ketones, acyloxymethyl ketones, O-acylhydroxylamines, vinyl sulfones and epoxysuccinic derivatives (S. Verhelst, M. Bogyo QSAR Comb. Sci. 2005, 24, 261-269).

To be effective as biological tools, protease inhibitors must be not only very potent but also highly selective in binding to a particular protease. The development of small molecule inhibitors for specific proteases has often started from peptide substrates. Although peptides display a diverse range of biological properties, their use as drugs can be compromised by their instability and their low oral bioavailability. To be effective drugs, protease inhibitors with reduced peptide-like character, high stability against non selective proteolytic degradation, high selectivity for a given protease, and good bioavailability to the locus of protease action are desirable. These requirements led to the development of protease inhibitors A-B with non-peptidic chemical scaffolds A, which are covalently linked to electrophilic warheads B. When bound to the protease, B reacts covalently with the catalytic residue (mechanism based inhibitor). In many cases the selectivity and pharmacokinetic properties of such inhibitors were successfully optimized in the context of biomedical research.

### DESCRIPTION OF THE INVENTION

The invention relates to molecular probes for proteases of the formula (I)

L1R1-L-A-X (I)

wherein

X is an electrophilic warhead; or X is a hydrogen;

A is a group recognizable by a protease;

R1 is a linker;

L is a bond or a group allowing for a facile conjugation of the group R1, and

L1 is a label optionally bound to a solid support.

The compounds of the formula (I) are imaging probes (inhibitors) for cysteine proteases, preferably from the cathepsin or caspase subfamilies, and for metalloproteases from the MMP or carboxypeptidase subfamilies.

In the case of cystein proteases, the warheads X react with the cystein residue in the active site resulting in a covalent attachment of the imaging probes to the enzyme and allowing further localization of the active protease. In the case of metalloproteases, the imaging probes bind to the active site of the protein through non-covalent forces e.g. hydrogen bonds, polar or Van der Waal's interactions.

In their most basic form, the imaging probe consists of four functional elements, a) an electrophilic warhead X as a reactive group, that can be attacked by a nucleophilic center of a protease, or a hydrogen b) a scaffold A which defines the selectivity for a given protease target, c) a linker moiety R1 to connect subunits to each other and d) a label L1 for detection.

Group A is preferably the main determinant for specificity towards a given protease or a group of proteases, preferably for the cathepsin K, S and B, e.g. as shown in compounds 1.-116. in Table 1-3, for caspase-1, -3 and -8, e.g. as shown in compounds 117.-157. in Table 4-6, for MMP's as shown in compounds 158 in Table 7 and for carboxypeptidases as shown in compounds 159.-167. in Table 8. Imaging probes of the present invention show selectivity for a given protease of the factor 1000 to 1, preferably a factor 10 to 1, wherein selectivity is defined by the relative inhibition (Ki with enzyme 1 versus Ki with enzyme 2) at a preferred inhibitor concentration. The relative inhibition is determined for each enzyme pair by dividing the Ki of the enzyme of interest (enzyme 1) by the Ki of another enzyme against which selectivity is desired (enzyme 2). For in vivo applications high selectivity is desired at low (e.g. micromolar or submicromolar) substrate concentrations.

For an imaging reagent of the present invention, where X represents an electrophilic warhead, Scheme 1 shows the reaction of a cysteine protease P with a substrate wherein A represents the specificity determinant, and P represents the protease with its reactive cysteine comprising the thiolate ion group S⁻:

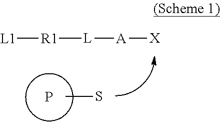

For an imaging reagent of the present invention, where X represents a hydrogen, Scheme 2 shows the binding of a given protease P to the labelling reagent wherein A represents the specificity determinant, and P represents the protease.

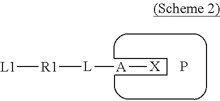

The reaction rate is dependent on the structure of the substrate.

L is a group selected from

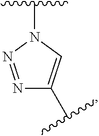

—(NRx)-, —O—, —C═N—, —C(═O)—, —C(═O)—NH—, —NH—C(═O)—, —C(═O)H, —CRx=CRy-, —C≡C— and phenyl, wherein Rx and Ry are independently H or (C₁-C₆)alkyl, or preferably a direct bond.

The linker group R1 is preferably a flexible linker connected to a label L1. The linker group is chosen in the context of the envisioned application, i.e. in context of an imaging probe for a specific protease. The linker may also increase the solubility of the substrate in the appropriate solvent. The linkers used are chemically stable under the conditions of the actual application. The linker does neither interfere with the reaction of a selected protease target nor with the detection of the label L1, but may be constructed such as to be cleaved at some point in time. More specifically, the linker group R1 is a straight or branched chain alkylene group with 1 to 300 carbon atoms, wherein optionally

(a) one or more carbon atoms are replaced by oxygen, in particular wherein every third carbon atom is replaced by oxygen, e.g. a polyethyleneoxy group with 1 to 100 ethyleneoxy units; and/or

(b) one or more carbon atoms are replaced by nitrogen carrying a hydrogen atom, and the adjacent carbon atoms are substituted by oxo, representing an amide function —NH—CO—; and/or

(c) one or more carbon atoms are replaced by an ester function —O—CO—;

(d) the bond between two adjacent carbon atoms is a double or a triple bond; and/or

(e) two adjacent carbon atoms are replaced by a disulfide linkage.

The label L1 of the substrate can be chosen by those skilled in the art dependent on the application for which the probe is intended.

The label L1 is a spectroscopic probe such as a fluorophore or a chromophore; a magnetic probe; a contrast reagent; a molecule which is one part of a specific binding pair which is capable of specifically binding to a partner; a molecule covalently attached to a polymeric support, a dendrimer, a glass slide, a microtiter plate known to those proficient in the art; or a molecule possessing a combination of any of the properties listed above.

The probe of the present invention can additionally comprise a targeting moiety such as an antibody, an antibody fragment, a receptor-binding ligand, a peptide fragment or a synthetic protein inhibitor.

Most preferred label L1 is a spectroscopic probe, furthermore an affinity label which is capable of specifically binding to a partner and molecules covalently attached to a solid support. An affinity label is defined as a molecule which is one part of a specific binding pair which is capable of specifically binding to a partner e.g. L1 is biotin binding to avidin or streptavidin or L1 is methotrexate, which is a tight-binding inhibitor of the enzyme dihydrofolate reductase (DHFR).

In particular, L1 is a fluorophore. Particularly preferred fluorophores are: a dimethylaminocoumarin derivative, preferably 7-dimethylaminocoumarin-4-acetic acid succinimidyl ester; Dansyl, 5/6-carboxyfluorescein, tetramethylrhodamine; difluoroboraindacenes, including Bodipy dyes as e.g. BODIPY-493/503, BODIPY-FL, BODIPY-TMR, BODIPY-TMR-X, BODIPY-TR-X, BODIPY630/550-X, BODIPY-650/665-X (U.S. Pat. No. 5,433,896); Alexa dyes, including Alexa 350, Alexa 488, Alexa 532, Alexa 546, Alexa 555, Alexa 635 and Alexa 647 (U.S. Pat. No. 5,696,157, U.S. Pat. No. 6,130,101, U.S. Pat. No. 6,716,979); Cy dyes, including Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5) (Amersham-GE Healthcare, Solingen, Germany); ATTO dyes, including ATTO 488, ATTO 532, ATTO 600, ATTO 655 (Atto-Tec, D57076 Siegen, Germany); Dy dyes, including DY-505, DY-547, DY-632, DY-647 (Dyomics, Jena, Germany).

Preferably, the compound of the formula (I) comprises a group X being an electrophilic warhead. More preferred, the compound of the formula (I) is a probe for proteases characterized by compounds comprising the following preferred warhead X:

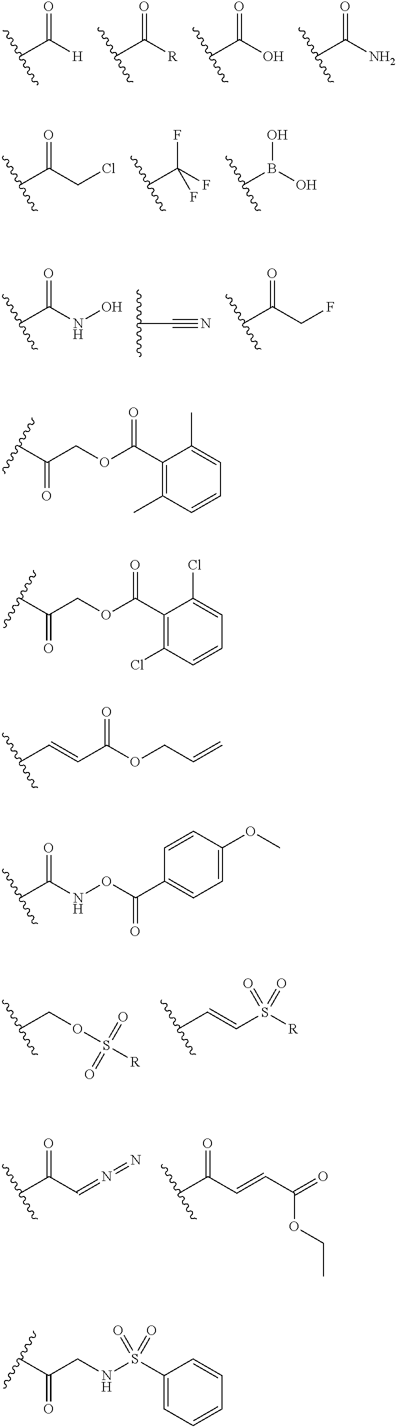

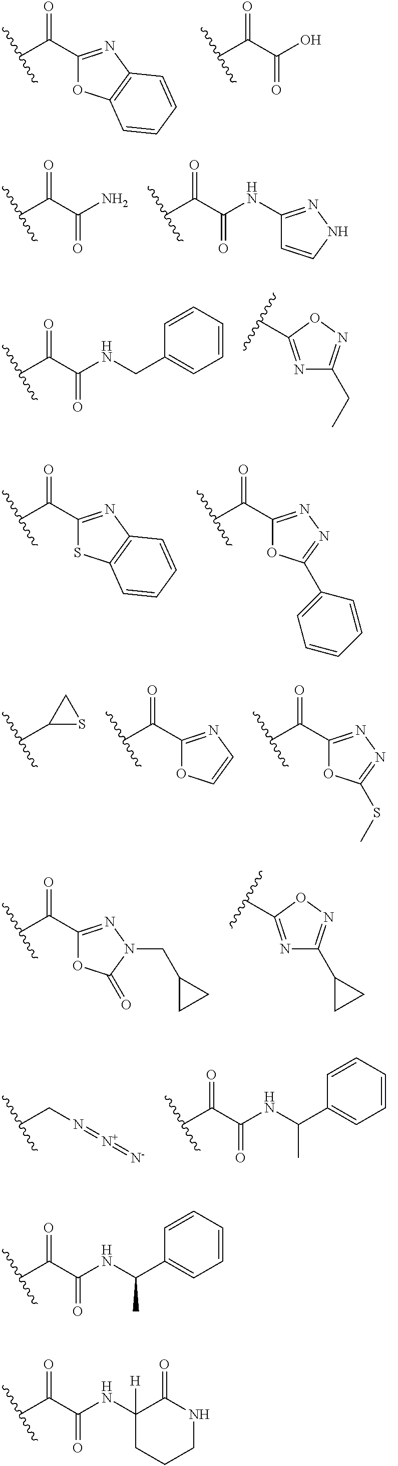

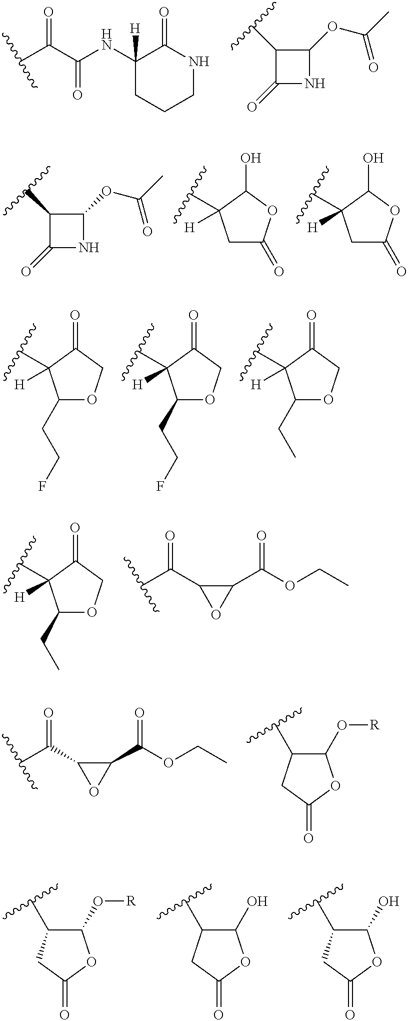

wherein R=alkyl, aryl.

Preferably, the compound of the formula (I) comprises a group A being an inhibitor of cathepsin K. International patent applications WO06076796, WO06076797, WO06063762 and WO05049028 disclose examples of selective cathepsin K inhibitors that may be used to be transformed into probes of the formula (I). More preferred, the compound of the formula (I) is a probe for cathepsin K characterized by a compound comprising the following preferred scaffolds A (Table 1):

TABLE 1
Examples of selective probes (I) for cathepsin K

| | |
|---|---|
| 1. | 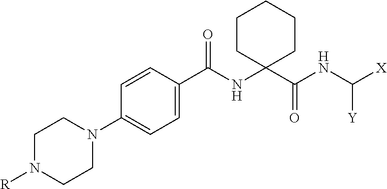 |
| 2. | 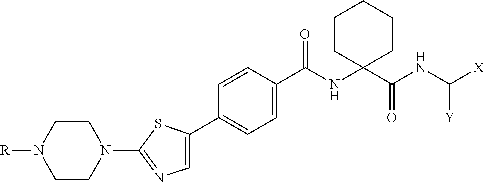 |
| 3. | 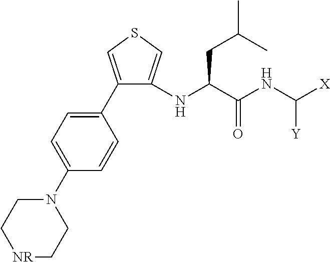 |
| 4. | 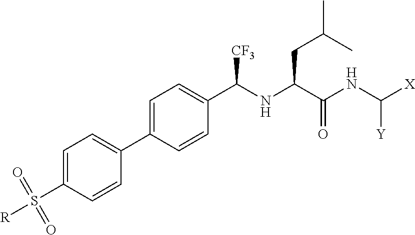 |
| 5. | 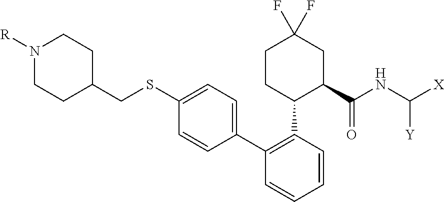 |
| 6. | 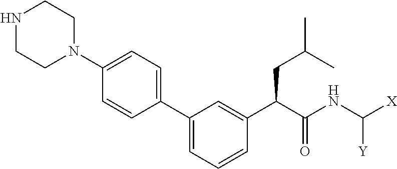 |
| 7. | 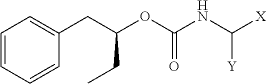 |
| 8. | 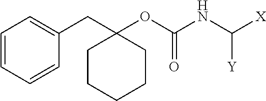 |
| 9. | 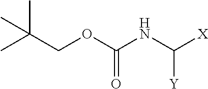 |
| 10. | 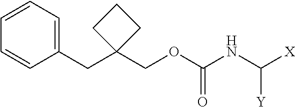 |
| 11. | 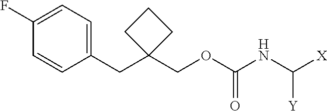 |
| 12. | 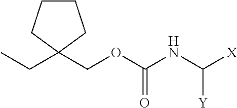 |
| 13. | 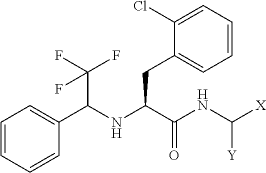 |
| 14. | 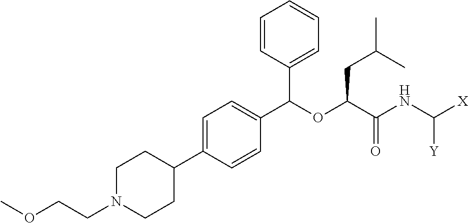 |
| 15. | 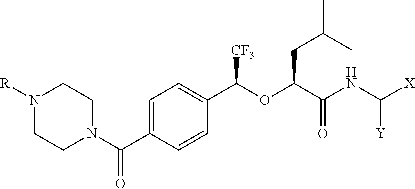 |
| 16. | 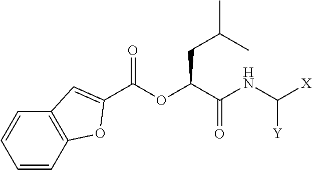 |
| 17. | 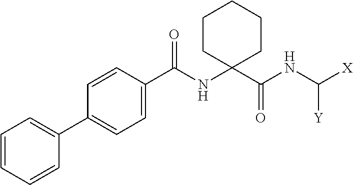 |
| 18. | 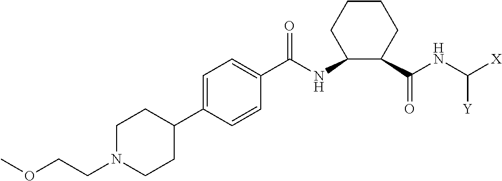 |
| 19. | 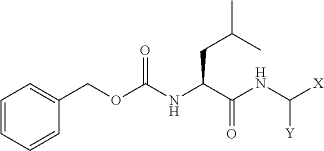 |
| 20. | 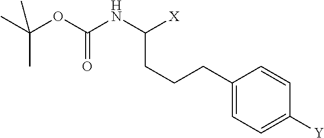 |
| 21. | 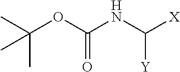 |
| 22. | 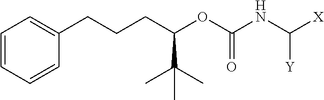 |
| 23. | 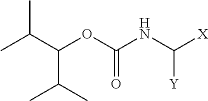 |
| 24. | 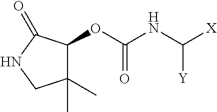 |
| 25. | 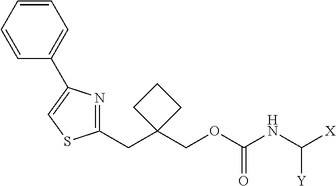 |
| 26. | 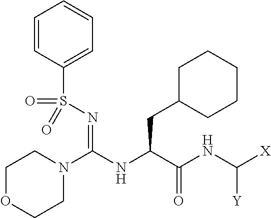 |
| 27. | 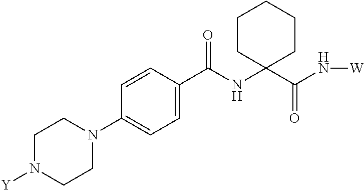 |
| 28. | 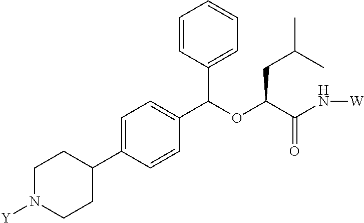 |
| 29. | 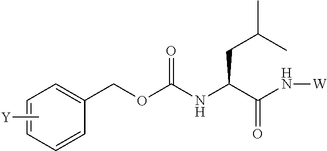 |
| 30. | 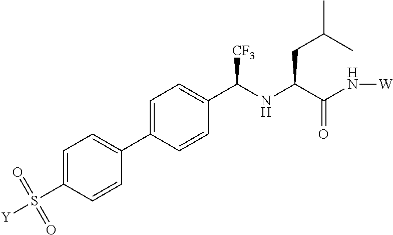 |
| 31. | 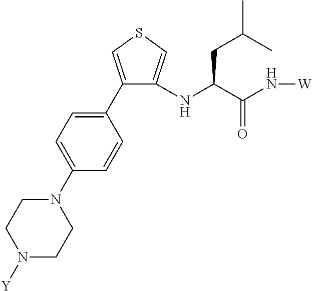 |
| 32. | 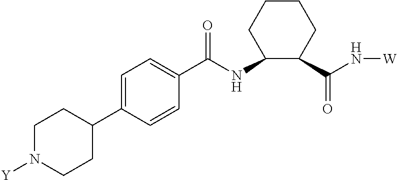 |
| 33. | 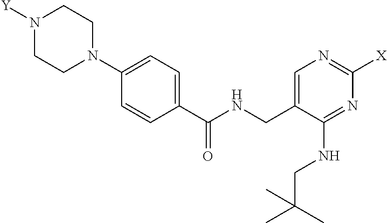 |
| 34. | 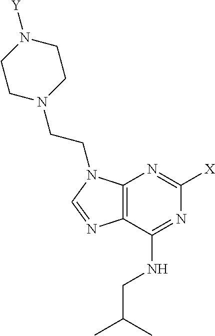 |
| 35. | 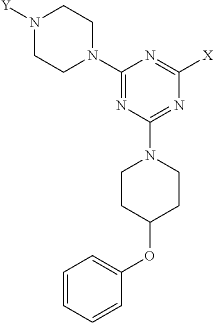 |
| 36. | 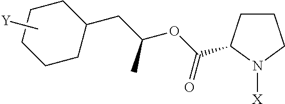 |
| 37. | 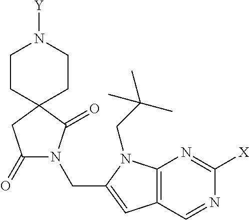 |
| 38. | 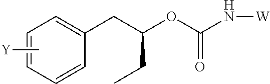 |
| 39. | 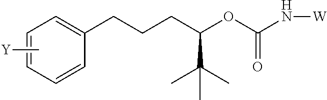 |
| 40. | 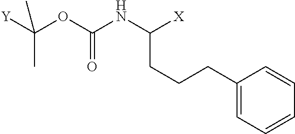 |
| 41. | 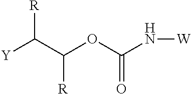 |
| 42. | 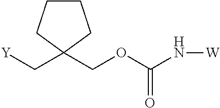 |
| 43. | 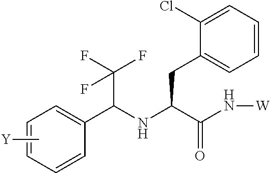 |
| 44. | 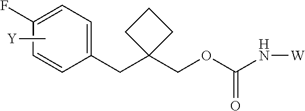 |
| 45. | 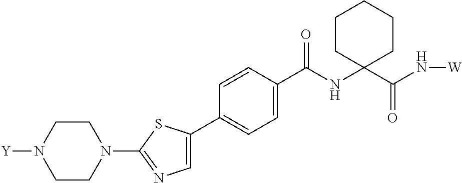 |
| 46. | 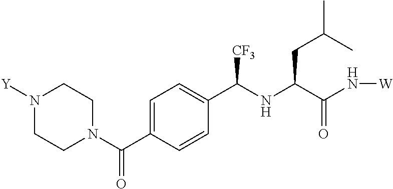 |
| 47. | 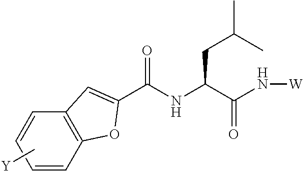 |
| 48. | 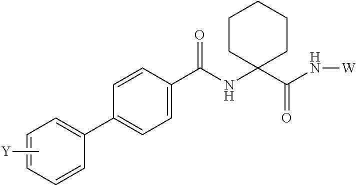 |
| 49. | 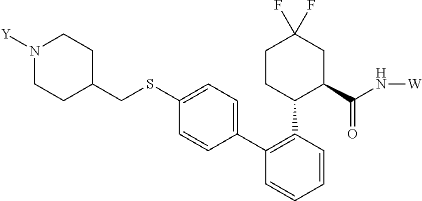 |
| 50. | 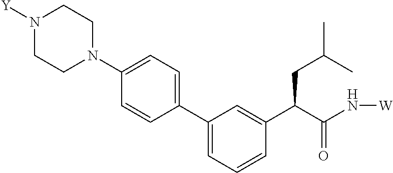 |
| 51. | 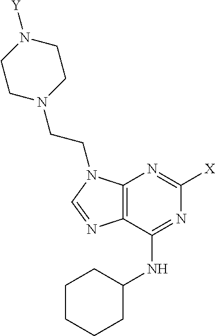 |
| 52. | 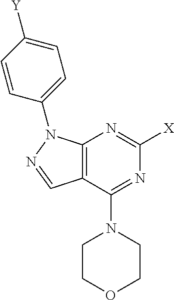 |
| 53. | 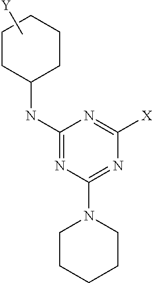 |
| 54. | 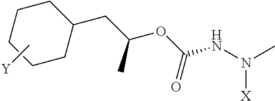 |
| 55. | 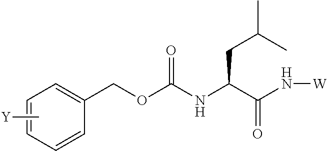 |
| 56. | 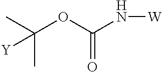 |
| 57. | 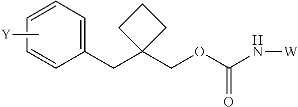 |
| 58. | 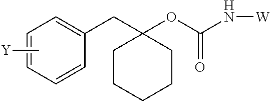 |
| 59. | 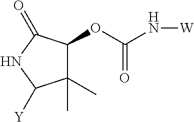 |
| 60. | 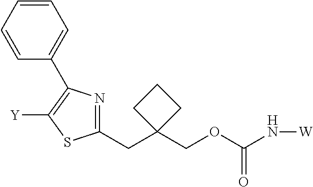 |
| 61. | 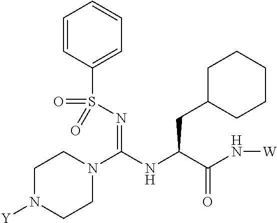 |

wherein independently of each other the variables in the compounds 1. to 61. are defined as indicated in the definition next to the respective compound; Y is -L-R1-L1; and R1, L and L1 are as described above; R is H or (C₁-C₆)-alkyl; and

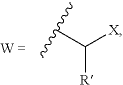

### or W═X,

wherein X is preferably defined as a group selected from

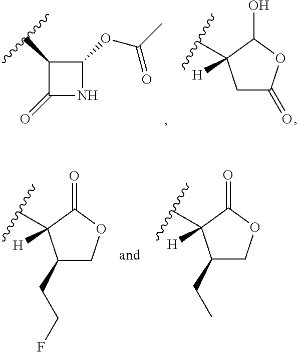

and R′ is H; or C₁-C₆-alkyl optionally substituted by

OH, —O—C₁-C₆-alkyl, —O—(C═O)—O—C₁-C₆-alkyl, SH, —S—C₁-C₆-alkyl, NH₂, —NH—C₁-C₆-alkyl, —N(C₁-C₆-alkyl)₂, —(C═O)—NH—C₁-C₆-alkyl, —COOH, —C(═O)O—C₁-C₆-alkyl or —NH—C(C═O)—C₁-C₆-alkyl; or aryl, preferably phenyl.

Compounds 1.-26. are substrates for cathepsin K with L1 in the S1 pocket, compounds 27.-61. for cathepsin K with L1 in the S3 pocket or beyond (outward).

Further preferably, the compound of the formula (I) comprises a group A being an inhibitor of cathepsin S. International patent applications WO04089395, WO0540142, WO0055144, WO05074904 and WO0069855 disclose examples of selective cathepsin S inhibitors that may be used to be transformed into probes of the formula (I). More preferred, the compound of the formula (I) is a probe for cathepsin S characterized by a compound comprising the following preferred scaffolds A (Table 2):

TABLE 2
Examples of selective probes (I) for cathepsin S

| | |
|---|---|
| 62. | 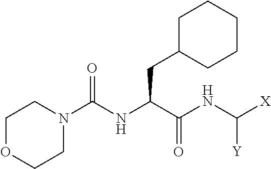 |
| 63. | 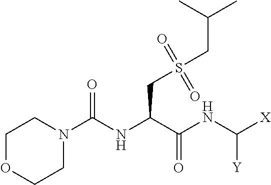 |
| 64. | 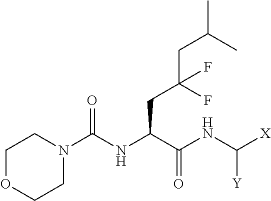 |
| 65. | 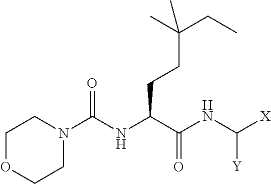 |
| 66. | 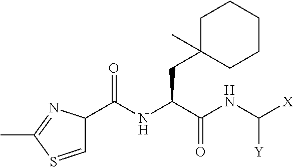 |
| 67. | 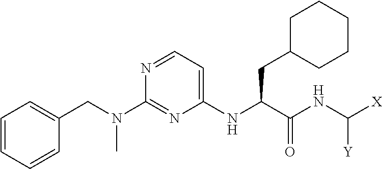 |
| 68. | 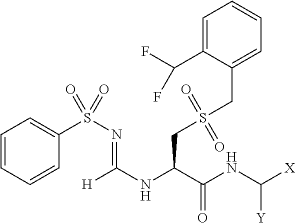 |
| 69. | 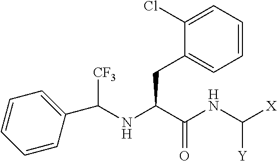 |
| 70. | 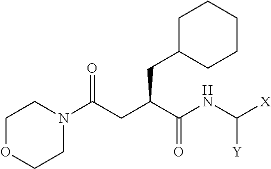 |
| 71. | 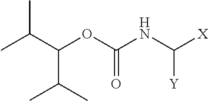 |
| 72. | 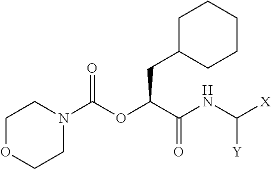 |
| 73. | 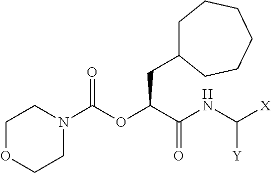 |
| 74. | 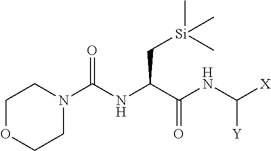 |
| 75. | 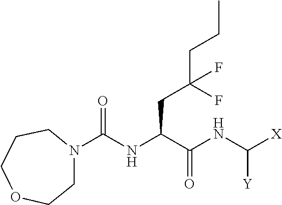 |
| 76. | 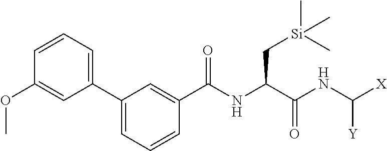 |
| 77. | 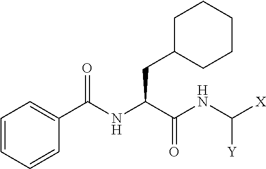 |
| 78. | 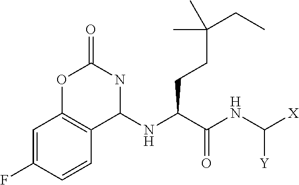 |
| 79. | 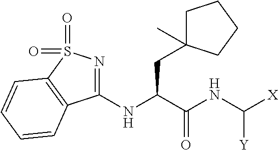 |
| 80. | 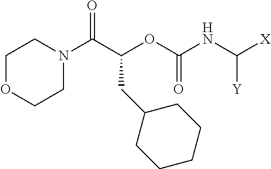 |
| 81. | 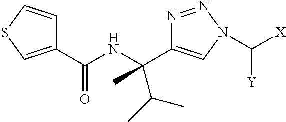 |
| 82. | 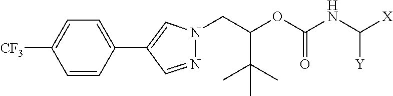 |
| 83. | 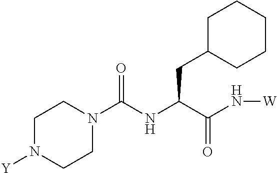 |
| 84. | 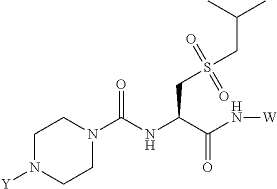 |
| 85. | 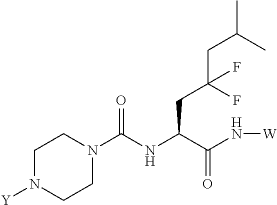 |
| 86. | 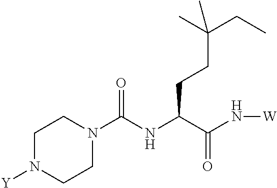 |
| 87. | 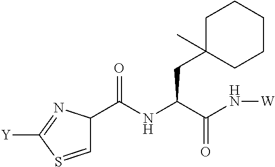 |
| 88. | 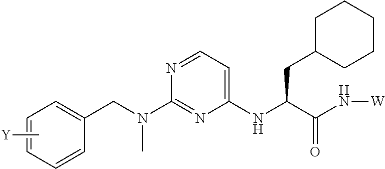 |
| 89. | 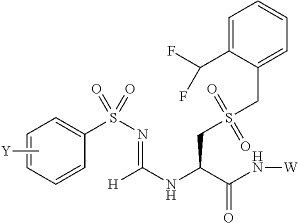 |
| 90. | 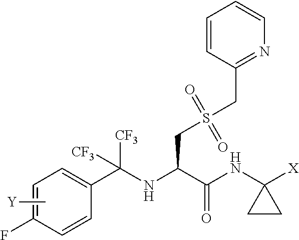 |
| 91. | 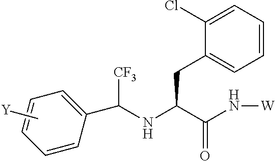 |
| 92. | 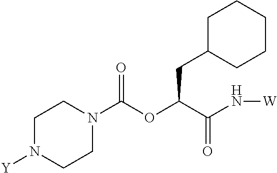 |
| 93. | 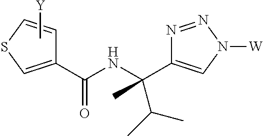 |
| 94. | 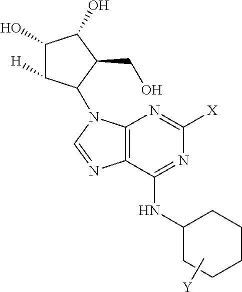 |
| 95. | 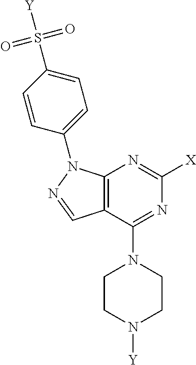 |
| 96. | 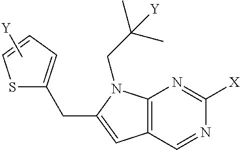 |
| 97. | 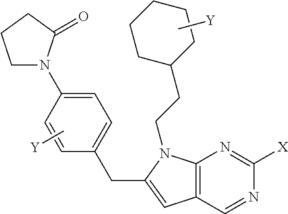 |
| 98. | 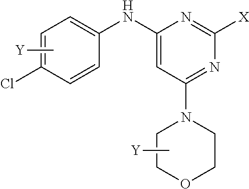 |
| 99. | 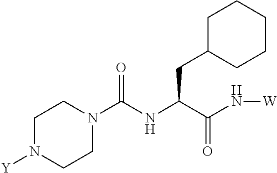 |
| 100. | 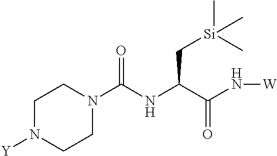 |
| 101. | 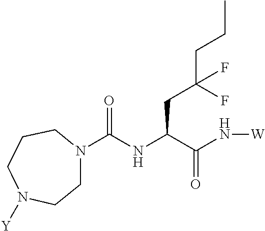 |
| 102. | 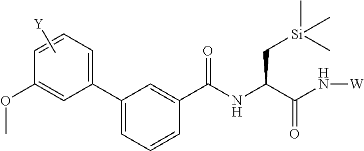 |
| 103. | 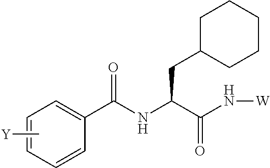 |
| 104. | 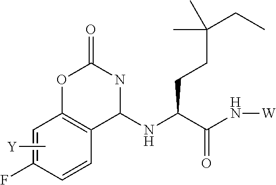 |
| 105. | 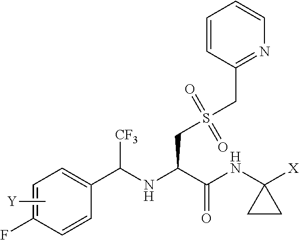 |
| 106. | 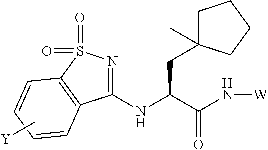 |
| 107. | 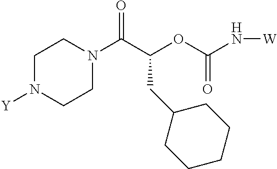 |
| 108. | 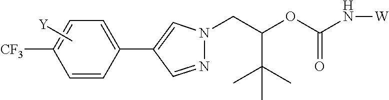 |
| 109. | 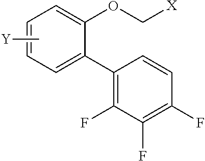 |
| 110. | 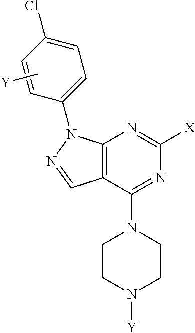 |
| 111. | 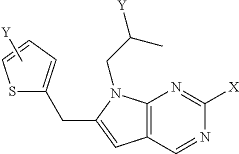 |
| 112. | 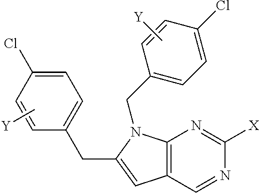 |
| 113. | 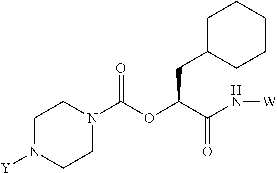 |
| 114. | 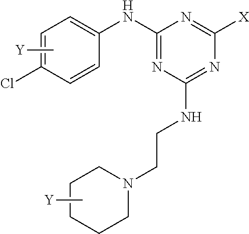 |

wherein independently of each other the variables in the compounds 62. to 114. are defined as indicated in the definition next to the respective compound; Y is -L-R1-L1; and R1, L and L1 are as described above;

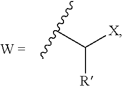

### or W═X,

wherein X is preferably defined as a group selected from

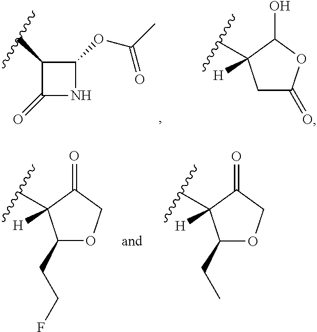

and R′ is H; or C₁-C₆-alkyl optionally substituted by

OH, —O—C₁-C₆-alkyl, —O—(C═O)—O—C₁-C₆-alkyl, SH, —S—C₁-C₆-alkyl, NH₂, —NH—C₁-C₆-alkyl, —N(C₁-C₆-alkyl)₂, —(C═O)—NH—C₁-C₆-alkyl, —COOH, —C(═O)O—C₁-C₆-alkyl or —NH—C(C═O)—C₁-C₆-alkyl; or aryl, preferably phenyl.

Compounds 62.-82. are substrates for cathepsin S with L1 in the S1 pocket, compounds 83.-114. for cathepsin S with L1 in the S3 pocket or beyond (outward).

Further preferably, the compound of the formula (I) comprises a group A being an inhibitor of cathepsin B. The preparation of scaffolds A having cathepsin B inhibitory activity is for example described in Greenspan et al. J. Med. Chem. 2001, 44, 4524-4534, and Greenspan et al. Bioorg. Med. Chem 2003, 13, 4121-4124. More preferred, the compound of the formula (I) is a probe for cathepsin B characterized by a compound comprising the following preferred scaffolds A (Table 3):

TABLE 3
Examples of selective probes (I) for cathepsin B

| | |
|---|---|
| 115. | 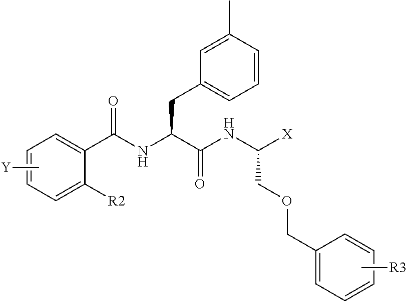 |
| | wherein R2 is a halogen and R3 is COOH or H. |
| 116. | 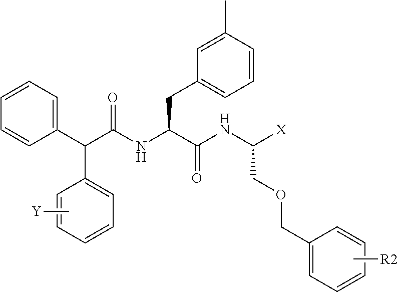 |
| | wherein R2 is COOH or H. |

wherein Y is -L-R1-L1; and R1, L and L1 are as described above.

Preferably, the compound of the formula (I) comprises a group A being an inhibitor of caspase-1. The preparation of scaffolds A having caspase-1 inhibitory activity is for example described in U.S. Pat. No. 5,670,494; WO9526958; WO9722619; WO9816504; WO0190063; WO03106460; WO03104231; WO03103677; W. G. Harter, Bioorg. Med. Chem. Lett. 2004, 14, 809-812; Shahripour et al., Bioorg. Med. Chem. Lett. 2001, 11, 2779-2782; Shahripour et al., Bioorg. Med. Chem. 2002, 10, 31-40; M. C. Laufersweiler et al., Bioorg. Med. Chem. Lett. 2005, 15, 4322-4326; K. T. Chapman, Bioorg. Med. Chem. Lett. 1992, 2, 613-618; Dolle et al., J. Med. Chem. 1997, 40, 1941-1946; D. L. Soper et al., Bioorg. Med. Chem. Lett. 2006, 16, 4233-4236; D. L. Soper et al., Bioorg. Med. Chem. 2006, 14, 7880-7892; D. J. Lauffer et al., Bioorg. Med. Chem. Lett. 2002, 12, 1225-1227; and C. D. Ellis et al., Bioorg. Med. Chem. Lett. 2006, 16, 4728-4732. More preferred, the compound of the formula (I) is a probe for caspase-1 characterized by a compound comprising the following preferred scaffolds A (Table 4):

TABLE 4
Examples of selective probes (I) for caspase-1

| | |
|---|---|
| 117. | 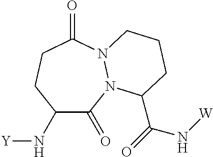 |
| | preferably in the configuration: |
| | 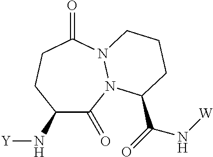 |
| 118. | 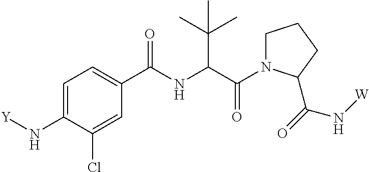 |
| | preferably in the configuration: |
| | 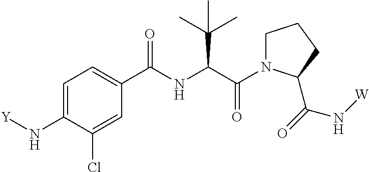 |
| 119. | 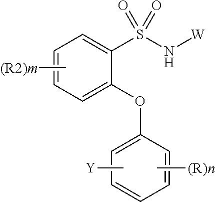 |
| | wherein |
| | R is (C₁-C₅)alkyl, phenyl, (C₅-C₆)cycloalkyl; |
| | R2 is alkyl: and |
| | n and m are independently of each other 0-3. |
| 120. | 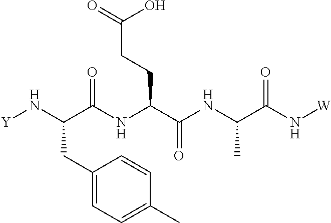 |
| 121. | 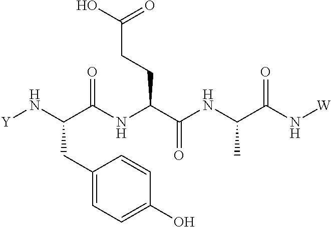 |
| 122. | 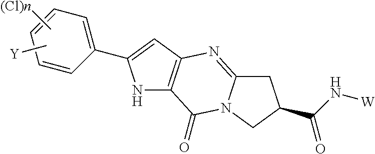 |
| | wherein n is 0 or 1; |
| 123. | 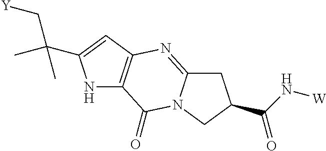 |
| 124. | 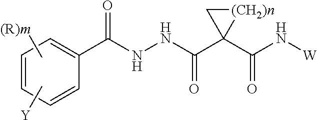 |
| | wherein |
| | n is 1-4; |
| | m is 1 or 2; and |
| | R is methyl or methoxy. |
| 125. | 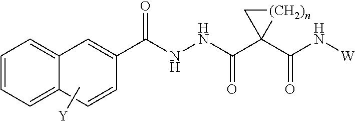 |
| | wherein n is 1-4. |
| 126. | 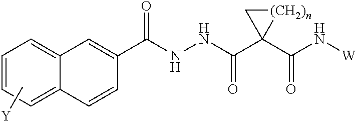 |
| | wherein n is 1-4. |
| 127. | 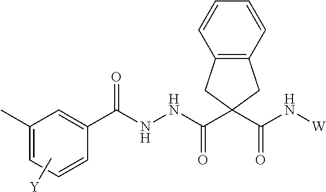 |
| 128. | 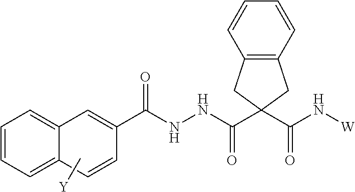 |
| 129. | 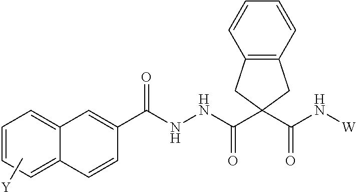 |
| 130. | 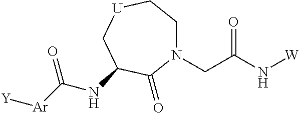 |
| | wherein |
| | U is S or S(O)₂; and |
| | Ar is aryl or heteroaryl, preferably phenyl, naphthyl, benzo- |
| | thiophene or isoquinolyl. |
| 131. | 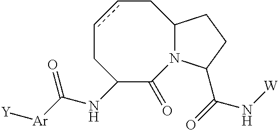 |
| | wherein |
| | Ar is an aryl or heteroaryl group selected from phenyl, benzo- |
| | thiophene, isoquinolyl, cinnamyl, naphthyl, which is optionally |
| | once or independently twice substituted by methoxy, chloro, |
| | methyl, CF₃, and wherein |
| | 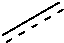 |
| | means either a single or a double bond. |
| 132. | 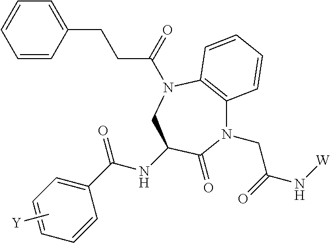 |
| 133. | 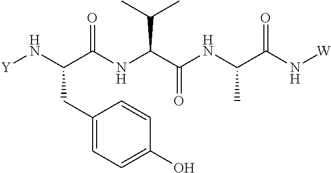 |
| 134. | 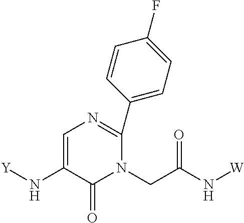 |
| 135. | 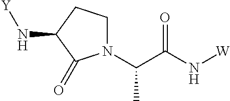 |
| 136. | 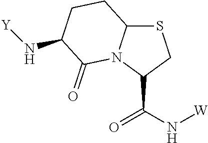 |
| 137. | 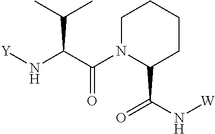 |
| 138. | 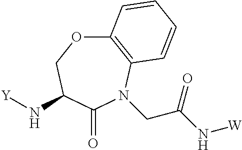 |
| 139. | 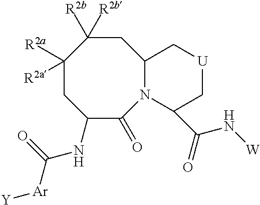 |
| | preferably in the all-(S) configuration, |
| | wherein |
| | Ar is aryl or heteroaryl; |
| | U is CH₂, O or NR9 wherein R9 is hydrogen or (C₁-C₆)alkyl, aryl, |
| | heteroaryl, heterocyclyl; |
| | R^{2a}, R^{2a′}, R^{2b} and R^{2b′} are each independently hydrogen, hydroxyl, |
| | N(R⁶)₂, halogen, (C₁-C₄)alkyl, (C₁-C₄)alkoxy, and mixtures thereof |
| | wherein R⁶ is hydrogen, (C₁-C₆)alkyl, cycloalkyl, (C₆-C₁₀)aryl; or |
| | R^{2a} and R^{2b} can taken together to form a double bond. |
| 140. | 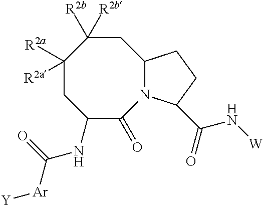 |
| | preferably in the all-(S) configuration, |
| | wherein |
| | Ar is aryl or heteroaryl; |
| | R^{2a}, R^{2a′}, R^{2b} and R^{2b′} are each independently hydrogen, hydroxyl, |
| | N(R⁶)₂, halogen, (C₁-C₄)alkyl, (C₁-C₄)alkoxy, and mixtures thereof |
| | wherein R⁶ is hydrogen, (C₁-C₆)alkyl, cycloalkyl, (C₆-C₁₀)aryl; or |
| | R^{2a} and R^{2b} can taken together to form a double bond. |
| 141. | 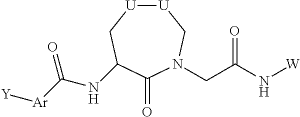 |
| | preferred in the all-(S) configuration |
| | wherein |
| | Ar is aryl or heteroaryl; and |
| | U is independently selected from: C(R¹)₂; C(O); NR²; S; S(O); |
| | S(O)₂; wherein R¹ and R² are independently hydrogen, |
| | [C(R³)₂]ₚ(CH═CH)_{q}R³, C(═Z)R³, C(═Z)[C(R³)₂]ₚ(CH═CH)_{q}R³, |
| | C(═Z)N(R³)₂, C(═Z)NR³N(R³)₂, CN, CF₃, N(R³)₂, NR³CN, |
| | NR³C(═Z)R³, NRC(═Z)N(R³)₂, NHN(R³)₂, NHOR³, NO₂, OR³, |
| | OCF₃, F, Cl, Br, I, SO₃H, OSO₃H, SO₂N(R³)₂, SO₂R³, P(O)(OR³)R³, |
| | P(O)(OR³)₂; wherein p is 0 to 12; wherein q is 0 to 12; wherein Z is |
| | O, S, NR³; wherein R³ is independently hydrogen, alkyl, cycloalkyl, |
| | aryl, heteroaryl, heterocyclyl. |
| 142. | 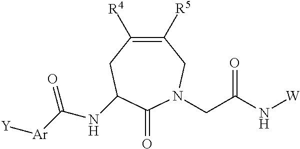 |
| | preferred in the all-(S) configuration |
| | wherein Ar is aryl or heteroaryl; and R⁴ and R⁵ are independently |
| | selected from: C(R¹)₂; C(O); NR²; S; S(O); S(O)₂; wherein R¹ and |
| | R² are independently hydrogen, [C(R³)₂]ₚ(CH═CH)_{q}R³, C(═Z)R³, |
| | C(═Z)[C(R³)₂]ₚ(CH═CH)_{q}R³, C(═Z)N(R³)₂, C(═Z)NR³N(R³)₂, |
| | CN, CF₃, N(R³)₂, NR³CN, NR³C(═Z)R³, NRC(═Z)N(R³)₂, |
| | NHN(R³)₂, NHOR³, NO₂, OR³, OCF₃, F, Cl, Br, I, SO₃H, OSO₃H, |
| | SO₂N(R³)₂, SO₂R³, P(O)(OR³)R³, P(O)(OR³)₂; wherein p is 0 to 12; |
| | wherein q is 0 to 12; wherein Z is O, S, NR³; wherein R³ is |
| | independently hydrogen, alkyl, cycloalkyl, aryl, heteroaryl, |
| | heterocyclyl. |

wherein independently of each other the variables in the compounds 117. to 142. are defined as indicated in the definition next to the respective compound; Y is -L-R1-L1; and R1, L and L1 are as described above; and

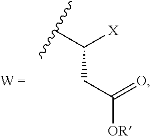

### or W═X,

wherein X is preferably a group selected from

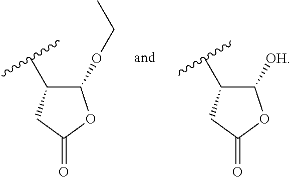

and R′ is H or C₁-C₆-alkyl.

Further preferably, the compound of the formula (I) comprises a group A being an inhibitor of caspase-3. The preparation of scaffolds A having caspase-3 inhibitory activity is for example described in WO0032620; WO0055127; WO0105772; WO03024955; P. Tawa et al., Cell Death and Differentiation 2004, 11, 439-447; Micale et al., J. Med. Chem. 2004, 47, 6455-6458; and Berger et al., Molecular Cell, 2006, 23, 509-521. More preferred, the compound of the formula (I) is a probe for caspase-3 characterized by a compound comprising the following preferred scaffolds A (Table 5):

TABLE 5
Examples of selective probes (I) for casapse-3

| | |
|---|---|
| 143. | 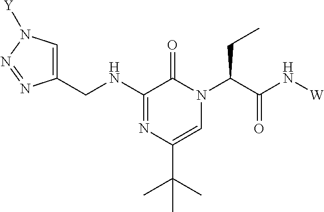 |
| 144. | 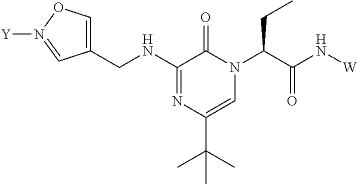 |
| 145 | 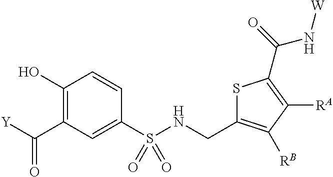 |
| | wherein R^{A} and R^{B} are independently hydrogen, (C₁-C₆)alkyl, |
| | hydroxyl, (C₁-C₆)alkoxy or halogen. |
| 146. | 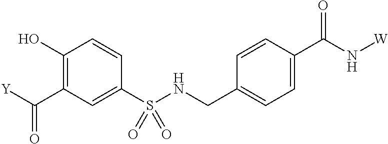 |
| 147. | 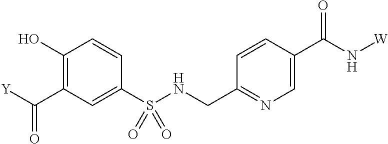 |
| 148. | 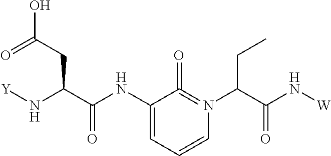 |
| 149. | 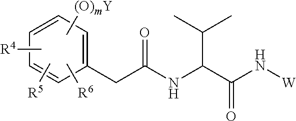 |
| | preferably in the all-(S) configuration, |
| | wherein |
| | m is 0 or 1; and |
| | R⁴, R⁵ and R⁶ are independently selected from the group consisting |
| | of: |
| | 1) H, |
| | 2) halogen, |
| | 3) (C₁-C₄)alkoxy optionally substituted with 1-3 halogen atoms, |
| | 4) NO₂, |
| | 5) OH, |
| | 6) benzyloxy, the benzyl portion of which is optionally substituted |
| | with 1-2 members selected from the group consisting of: halogen, |
| | CN, (C₁-C₄)alkyl and (C₁-C₄)alkoxy, said alkyl and alkoxy being |
| | optionally substituted with 1-3 halogen groups, |
| | 7) NH(C₁-C₄)acyl, |
| | 8) (C₁-C₄)acyl, |
| | 9) O—(C₁-C₄)alkyl-CO₂H, optionally esterified with a (C₁-C₆)alkyl |
| | or a (C₅-C₇)cycloalkyl group, |
| | 10) CH═CH—CO₂H, |
| | 11) CO₂H, |
| | 12) (C₁-C₅)alkyl-CO₂H, |
| | 13) C(O)NH₂, optionally substituted on the nitrogen atom by 1-2 |
| | (C₁-C₄)alkyl groups; |
| | 14) (C₁-C₅)alkyl-C(O)NH₂, optionally substituted on the nitrogen |
| | atom by 1-2 (C₁-C₄)alkyl groups; |
| | 15) S(O)₀₋₂—(C₁-C₄)alkyl; |
| | 16) (C₁-C₂)alkyl-S(O)₀₋₂—(C₁-C₄)alkyl; |
| | 17) S(O)₀₋₂—(C₁-C₆)alkyl or S(O)₀₋₂-phenyl, said alkyl and phenyl |
| | portions thereof being optionally substituted with 1-3 members |
| | selected from the group consisting of: halogen, CN, (C₁-C₄)alkyl and |
| | (C₁-C₄)alkoxy, said alkyl and alkoxy being optionally substituted by |
| | 1-3 halogen groups, |
| | 18) benzoyl optionally substituted by 1-2 members selected from the |
| | group consisting of: halogen, CN, (C₁-C₄)alkyl and (C₁-C₄)alkoxy, |
| | said alkyl and alkoxy groups being optionally substituted by 1-3 |
| | halogen groups, |
| | 19) phenyl or naphthyl, optionally substituted with 1-2 members |
| | selected from the group consisting of: halogen, CN, (C₁-C₄)alkyl and |
| | (C₁-C₄)alkoxy, said alkyl and alkoxy being optionally substituted |
| | with 1-3 halogen groups, |
| | 20) CN, |
| | 21) (C₁-C₄)alkylene-HET2, wherein HET2 represents a 5-7 |
| | membered aromatic or non-aromatic ring containing 1-4 heteroatoms selected from O, S, NH and N(C₁-C₄) and optionally containing 1-2 |
| | oxo groups, and optionally substituted with 1-3 (C₁-C₄)alkyl, OH, |
| | halogen or (C₁-C₄)acyl groups; |
| | 22) O—(C₁-C₄)alkyl-HET3, wherein HET3 is a 5 or 6 membered |
| | aromatic or non-aromatic ring containing from 1 to 3 heteroatoms |
| | selected from O, S and N, and optionally substituted with one or two |
| | groups selected from halogen and (C₁-C₄)alkyl, and optionally |
| | containing 1-2 oxo groups, and |
| | 23) HET4, wherein HET4 is a 5 or 6 membered aromatic or non- |
| | aromatic ring, and the benzofused analogs thereof, containing from 1 |
| | to 4 heteroatoms selected from O, S and N, and is optionally |
| | substituted by one or two groups selected from halogen, (C₁-C₄)alkyl |
| | and (C₁-C₄)acyl; and wherein halogen includes F, Cl, Br and I. |
| 150. | 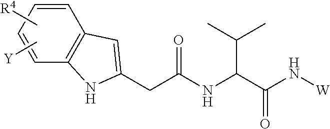 |
| | preferably in the all-(S) configuration, |
| | wherein R⁴ is selected from the group consisting of: |
| | 1) H, |
| | 2) halogen, |
| | 3) (C₁-C₄)alkoxy optionally substituted with 1-3 halogen atoms, |
| | 4) NO₂, |
| | 5) OH, |
| | 6) benzyloxy, the benzyl portion of which is optionally substituted |
| | with 1-2 members selected from the group consisting of: halogen, |
| | CN, (C₁-C₄)alkyl and (C₁-C₄)alkoxy, said alkyl and alkoxy being |
| | optionally substituted with 1-3 halogen groups, |
| | 7) NH(C₁-C₄)acyl, |
| | 8) (C₁-C₄)acyl, |
| | 9) O—(C₁-C₄)alkyl-CO₂H, optionally esterified with a (C₁-C₆)alkyl |
| | or a (C₅-C₇)cycloalkyl group, |
| | 10) CH═CH—CO₂H, |
| | 11) CO₂H, |
| | 12) (C₁-C₅)alkyl-CO₂H, |
| | 13) C(O)NH₂, optionally substituted on the nitrogen atom by 1-2 |
| | (C₁-C₄)alkyl groups; |
| | 14) (C₁-C₅)alkyl-C(O)NH₂, optionally substituted on the nitrogen |
| | atom by 1-2 (C₁-C₄)alkyl groups; |
| | 15) S(O)₀₋₂—(C₁-C₄)alkyl; |
| | 16) (C₁-C₂)alkyl-S(O)₀₋₂—(C₁-C₄)alkyl; |
| | 17) S(O)₀₋₂—(C₁-C₆)alkyl or S(O)₀₋₂-phenyl, said alkyl and phenyl |
| | portions thereof being optionally substituted with 1-3 members |
| | selected from the group consisting of: halogen, CN, (C₁-C₄)alkyl and |
| | (C₁-C₄)alkoxy, said alkyl and alkoxy being optionally substituted by |
| | 1-3 halogen groups, |
| | 18) benzoyl optionally substituted by 1-2 members selected from the |
| | group consisting of: halogen, CN, (C₁-C₄)alkyl and (C₁-C₄) alkoxy, |
| | said alkyl and alkoxy groups being optionally substituted by 1-3 |
| | halogen groups, |
| | 19) phenyl or naphthyl, optionally substituted with 1-2 members |
| | selected from the group consisting of: halogen, CN, (C₁-C₄)alkyl and |
| | (C₁-C₄)alkoxy, said alkyl and alkoxy being optionally substituted |
| | with 1-3 halogen groups, |
| | 20) CN, |
| | 21) (C₁-C₄)alkylene-HET2, wherein HET2 represents a 5-7 |
| | membered aromatic or non-aromatic ring containing 1-4 heteroatoms |
| | selected from O, S, NH and N(C₁-C₄) and optionally containing 1-2 |
| | oxo groups, and optionally substituted with 1-3 (C₁-C₄)alkyl, OH, |
| | halogen or (C₁-C₄)acyl groups; |
| | 22) O—(C₁-C₄)alkyl-HET3, wherein HET3 is a 5 or 6 membered |
| | aromatic or non-aromatic ring containing from 1 to 3 heteroatoms |
| | selected from O, S and N, and optionally substituted with one or two |
| | groups selected from halogen and (C₁-C₄)alkyl, and optionally |
| | containing 1-2 oxo groups, and |
| | 23) HET4, wherein HET4 is a 5 or 6 membered aromatic or non- |
| | aromatic ring, and the benzofused analogs thereof, containing from 1 |
| | to 4 heteroatoms selected from O, S and N, and is optionally |
| | substituted by one or two groups selected from halogen, (C₁-C₄)alkyl and (C₁-C₄)acyl; and wherein halogen includes F, Cl, Br and I. |
| 151. | 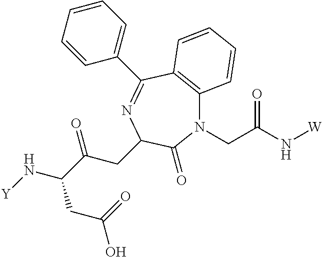 |
| 152. | 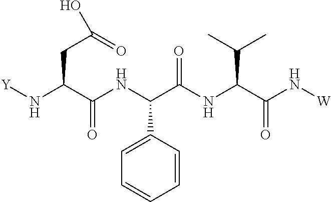 |
| 153. | 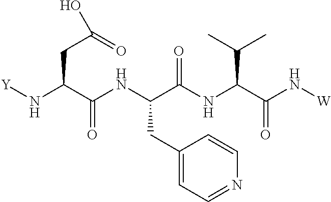 |
| 154. | 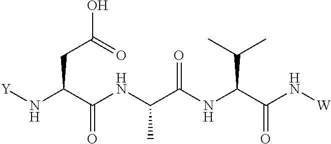 |
| 155. | 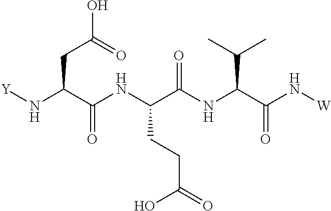 |

wherein independently of each other the variables in the compounds 143. to 155. are defined as indicated in the definition next to the respective compound; Y is -L-R1-L1; and R1, L and L1 are as described above; and

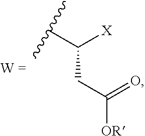

### or W═X,

wherein X is preferably a group selected from

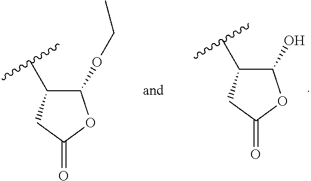

and R′ is H or C₁-C₆-alkyl.

Further preferably, the compound of the formula (I) comprises a group A being an inhibitor of caspase-8. The preparation of scaffolds A having caspase-8 inhibitory activity is for example described in Berger et al., Molecular Cell, 2006, 23, 509-521; and Garcia-Calvo, J. Biol. Chem. 1998, 273, 32608-32613. More preferred, the compound of the formula (I) is a probe for caspase-8 characterized by a compound comprising the following preferred scaffolds A (Table 6):

TABLE 6
Examples of selective probes (I) for caspase-8

| | |
|---|---|
| 156. | 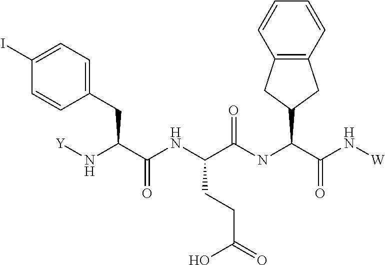 |
| 157. | 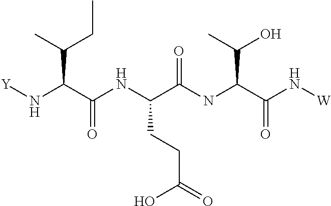 |

wherein independently of each other the variables in the compounds 156. to 157. are defined as indicated in the definition next to the respective compound; Y is -L-R1-L1; and R1, L and L1 are as described above; and

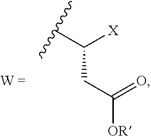

### or W═X,

wherein X is preferably a group selected from

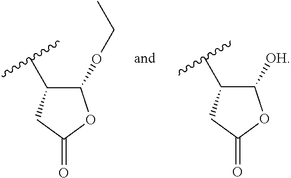

and R′ is H or C₁-C₆-alkyl.

Further preferably, the compound 158. comprises a group A being an inhibitor of MMP-13. The properties of scaffolds A having MMP-13 inhibitory activity is for example described in K. U. Wendt, C. K. Engel et al. Chemistry & Biology, 2005, 12, 181-189.

More preferred, the compound 158. is a probe for MMP-13 characterized by a compound comprising the following preferred scaffolds A (Table 7):

TABLE 7
Examples of selective probes (I) for MMP-13

| | |
|---|---|
| 158. | 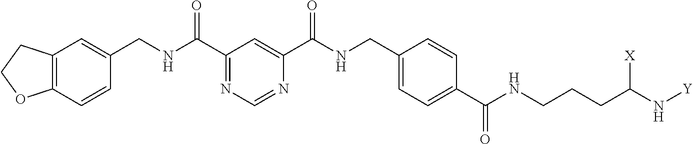 |

wherein Y is -L-R1-L1; and R1, L and L1 are as described above and X is a hydrogen.

Further preferred compounds 159.-167. comprise a group A being an inhibitor of carboxypeptidase U [Thrombin activable fibrinolysis inhibitor (TAFI)]. The scaffolds A having TAFI inhibitory activity are disclosed in M. E. Bunnage et al. J. Med. Chem. 2007, 50(24), 6095-6103, S. Gruneberg QSAR Comb. Sci. 2005, 24, 517-526, DE102005049385, WO0214285, WO05105781 and US2006234986.

TABLE 8
Examples of selective probes (I) for TAFI

| | |
|---|---|
| 159. | 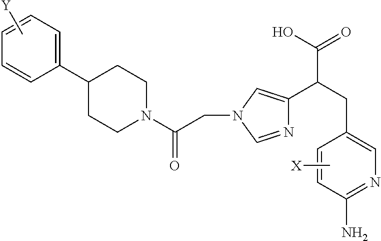 |
| 160. | 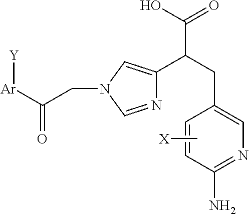 |
| | wherein Ar is aryl. |
| 161. | 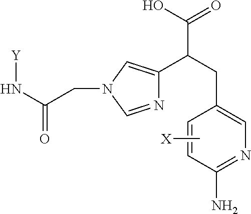 |
| 162. | 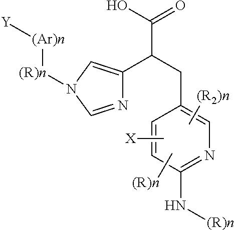 |
| | wherein R is alkyl, cycloalkyl; R₂ is halogen; Ar is aryl; and each n is |
| | independently 0 or 1. |
| 163. | 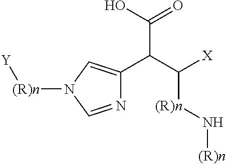 |
| | wherein R is alkyl, cycloalkyl; and each n is independently 0 or 1. |
| 164. | 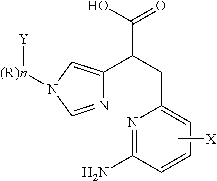 |
| | wherein R is alkyl, cycloalkyl; and n is 0 or 1. |
| 165. | 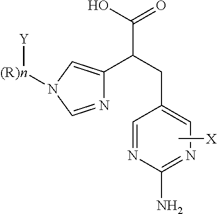 |
| | wherein R is alkyl, cycloalkyl; and n is 0 or 1. |
| 166. | 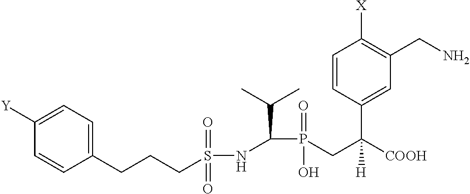 |
| 167. | 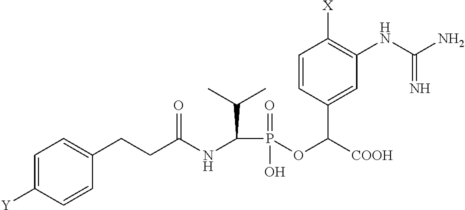 |

wherein Y is -L-R1-L1; and R1, L and L1 are as described above, and X is a hydrogen atom.

The compounds shown in Tables 1 to 8 show preferred compounds of the formula (I) comprising preferred groups A, i.e. groups Y (L1R1-L) and X are not shown in the said Tables.

More preferred, the invention relates to a molecular probe of the formula (I) wherein

A is a group as shown in Tables 1 to 8;

L is a direct bond or a group selected from

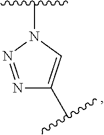

—(NRx)-, —O—, —C═N—, —C(═O)—, —C(═O)—NH—, —NH—C(═O)—, —C(═O)H, —CRx=CRy-, —C≡C— and phenyl, wherein Rx and Ry are independently H or (C₁-C₆)alkyl;

R1 is a straight or branched chain alkylene group with 1 to 300 carbon atoms, wherein optionally

(a) one or more carbon atoms are replaced by oxygen, in particular wherein every third carbon atom is replaced by oxygen, e.g. a polyethyleneoxy group with 1 to 100 ethyleneoxy units; and/or

(b) one or more carbon atoms are replaced by nitrogen carrying a hydrogen atom, and the adjacent carbon atoms are substituted by oxo, representing an amide function —NH—CO—; and/or

(c) one or more carbon atoms are replaced by an ester function —O—CO—;

(d) the bond between two adjacent carbon atoms is a double or a triple bond; and/or

(e) two adjacent carbon atoms are replaced by a disulfide linkage.

More preferred, R1 alkyl or a straight or branched chain alkylene group with 1 to 50 carbon atoms, wherein one or more carbon atoms are replaced by oxygen, in particular wherein every third carbon atom is replaced by oxygen, most preferred a polyethyleneoxy group with 1 to 20 ethyleneoxy units (polyethylene glycol, PEG).

L1 is biotin or methotrexate or a fluorophore selected from the group consisting of a dimethylaminocoumarin derivative, preferably 7-dimethylaminocoumarin-4-acetic acid succinimidyl ester, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647; most preferred L1 is a fluorophore selected from the group consisting of a dimethylaminocoumarin derivative, preferably 7-dimethylaminocoumarin-4-acetic acid succinimidyl ester, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647; and

X is a group selected from

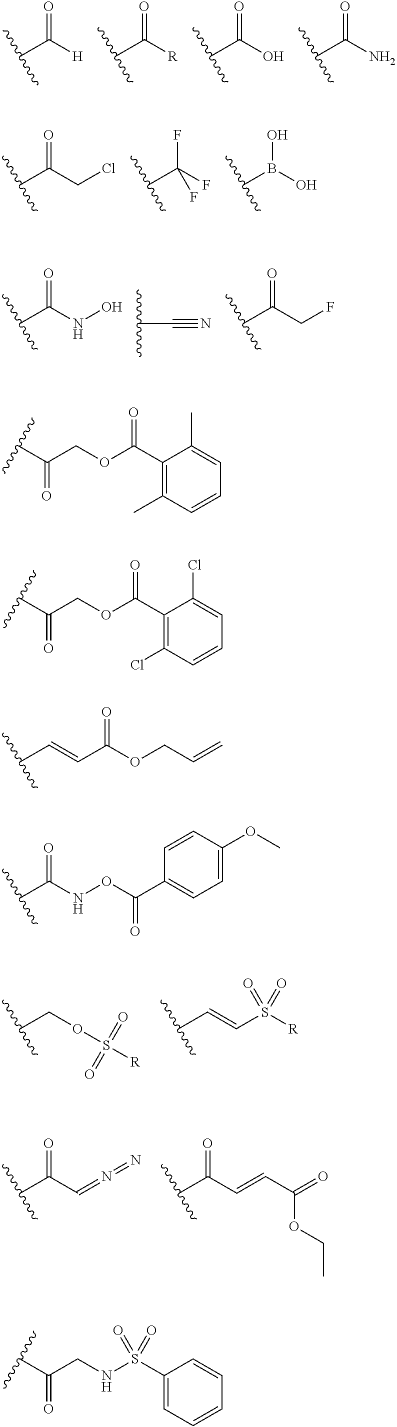

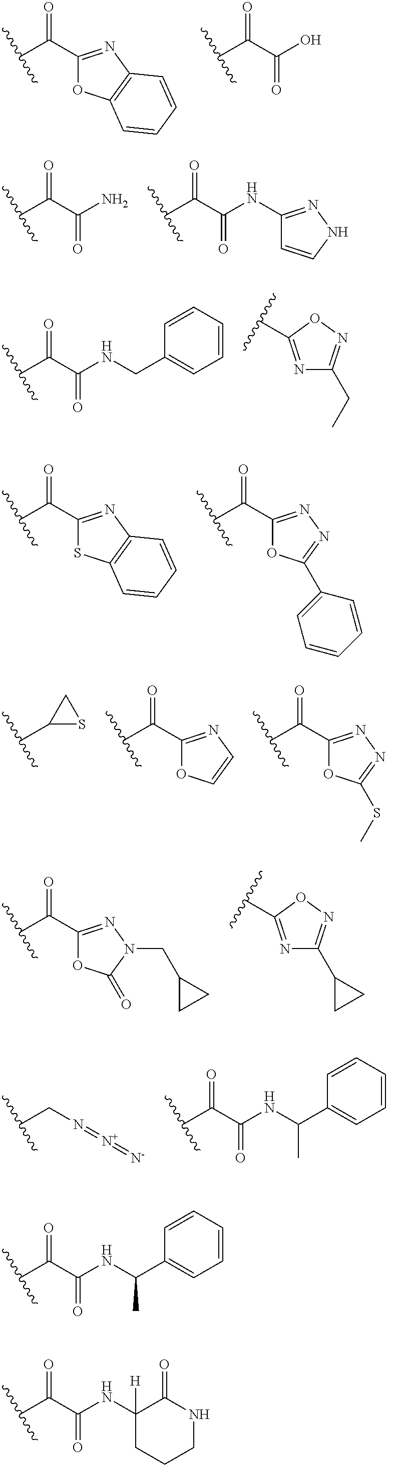

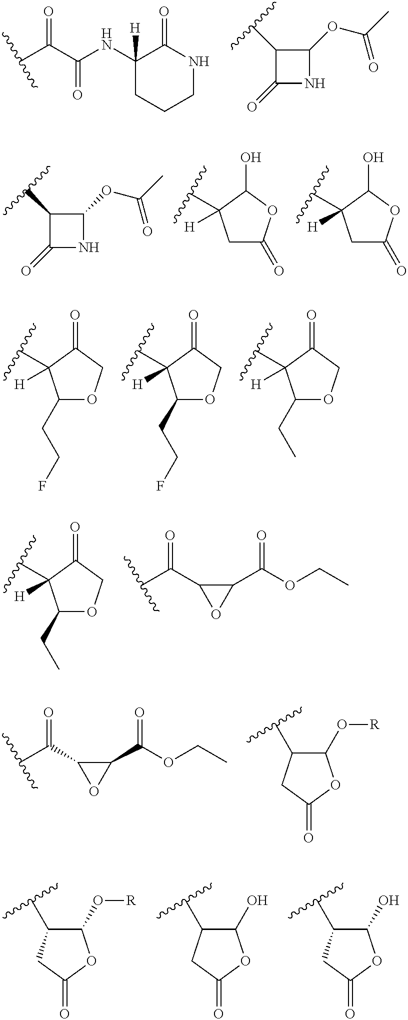

wherein R is alkyl, aryl.

More preferred, the invention relates to molecular probes for proteases of the formula (I) wherein

A is a group as shown Tables 1 to 8;

L is a group selected from

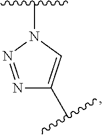

—(NRx)-, —O—, —C═N—, —C(═O)—, —C(═O)—NH—, —NH—C(═O)—, —C(═O)H, —CRx=CRy-, —C≡C— and phenyl, wherein Rx and Ry are independently H or (C₁-C₆)alkyl, or preferably a direct bond;

R1 is alkyl or a straight or branched chain alkylene group with 1 to 50 carbon atoms, wherein one or more carbon atoms are replaced by oxygen, in particular wherein every third carbon atom is replaced by oxygen, most preferred a polyethyleneoxy group with 1 to 20 ethyleneoxy units (polyethylene glycol, PEG);

L1 is a fluorophore selected from the group consisting of a dimethylaminocoumarin derivative, preferably 7-dimethylaminocoumarin-4-acetic acid succinimidyl ester, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647; more preferred L1 is a dimethylaminocoumarin derivative, preferably 7-dimethylaminocoumarin-4-acetic acid succinimidyl ester, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647;

X is a nitrile group or a group selected from

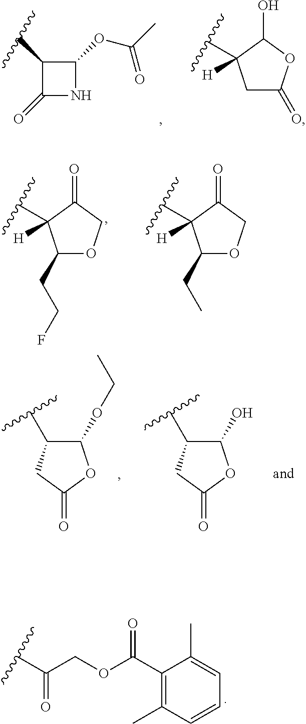

If not otherwise indicated, the terms alkyl, alkylene, cycloalkyl, heterocyclyl, aryl and heteroaryl are defined as follows:

The terms alkyl and alkylene are understood as a hydrocarbon residue having, if not indicated otherwise, 1 to 6 carbon atoms which can be linear, i.e. straight-chain, or branched. This also applies if an alkyl group occurs as a substituent on another group, for example in an alkoxy group (O-alkyl). Examples of alkyl groups as may be present are methyl, ethyl, n-propyl, n-butyl, n-pentyl, n-hexyl, isopropyl, isobutyl, 1-methylbutyl, isopentyl, neopentyl, 2,2-dimethylbutyl, 2-methylpentyl, 3-methylpentyl, isohexyl, sec-butyl, tert-butyl or tert-pentyl.

Cycloalkyl groups are cyclic alkyl groups containing, if not indicated otherwise, 3 to 8 ring carbon atoms, for example cyclopropyl, cyclobutyl, cyclopentyl, cyclohexyl or cyclooctyl.

Aryl groups mean (i) an aromatic ring or (ii) an aromatic ring system which comprises two aromatic rings which are fused or otherwise linked, that may be partly saturated and contain, if not indicated otherwise, 6 to 10 carbon atoms, for example phenyl, naphthyl, biphenyl, tetrahydronaphthyl, alpha- or beta-tetralon-, indanyl- or indan-1-on-yl group.

Heterocyclyl group means a 4-10 membered mono- or bicyclic ring system which comprises, apart from carbon, one or more heteroatoms such as, for example, e.g. 1, 2, 3 or 4 nitrogen atoms, 1 or 2 oxygen atoms, 1 or 2 sulfur atoms or combinations of different hetero atoms. For example, a C₆-heterocyclyl may contain 5 carbon atoms and 1 nitrogen atom as is the case in pyridyl or piperidinyl. The heterocyclyl residues can be bound at any positions, for example on the 1-position, 2-position, 3-position, 4-position, 5-position, 6-position, 7-position or 8-position. Heterocyclyl encompasses (i) heteroaryl groups, (ii) saturated heterocyclyl groups and (iii) mixed aromatic/saturated fused (C₈-C₁₀)heterocyclyl groups. Suitable heterocyclyl group include acridinyl, azetidine, benzimidazolyl, benzofuryl, benzomorpholinyl, benzothienyl, benzothiophenyl, benzoxazolyl, benzthiazolyl, benztriazolyl, benztetrazolyl, benzisoxazolyl, benzisothiazolyl, carbazolyl, 4aH-carbazolyl, carbolinyl, furanyl, quinazolinyl, quinolinyl, 4H-quinolizinyl, quinoxalinyl, quinuclidinyl, chromanyl, chromenyl, cinnolinyl, decahydroquinolinyl, 2H,6H-1,5,2-dithiazinyl, dihydrofuro[2,3-b]-tetrahydrofuran, furyl, furazanyl, homomorpholinyl, homopiperazinyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, prolinyl, pteridinyl, purynyl, pyranyl, pyrazinyl, pyroazolidinyl, pyrazolinyl, pyrazolyl, pyridazinyl, pyridonyl, pyridooxazoles, pyridoimidazoles, pyridothiazoles, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, 6H-1,2,5-thiadazinyl, thiazolyl, 1,2,3-thiadiazolyl, 1,2,4-thiadiazolyl, 1,2,5-thiadiazolyl, 1,3,4-thiadiazolyl, thienyl, triazolyl, tetrazolyl and xanthenyl. Pyridyl stands both for 2-, 3- and 4-pyridyl. Thienyl stands both for 2- and 3-thienyl. Furyl stands both for 2- and 3-furyl. Also included are the corresponding N-oxides of these compounds, for example, 1-oxy-2-, 3- or 4-pyridyl. Preferred examples of (C₄-C₁₀)heterocyclyl residues are 2- or 3-thienyl, 2- or 3-furyl, 1-, 2- or 3-pyrrolyl, 1-, 2-, 4- or 5-imidazolyl, 1-, 3-, 4- or 5-pyrazolyl, 1,2,3-triazol-1-, -4 or -5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4 or -5-yl, 1,2,4-oxadiazol-3 or -5-yl, 1,3,4-oxadiazol-2-yl or -5-yl, 2-, 4- or 5-thiazolyl, 3-, 4- or 5-isothiazolyl, 1,3,4-thiadiazol-2 or -5-yl, 1,2,4-thiadiazol-3 or -5-yl, 1,2,3-thiadiazol-4- or -5-yl, 2-, 3- or 4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-chinolyl, 1-, 3-, 4-, 5-, 6-, 7- or 8-isochinolyl, 2-, 4-, 5-, 6-, 7- or 8-chinazolinyl, 3-, 4-, 5-, 6-, 7- or 8-cinnolinyl, 2-, 3-, 5-, 6-, 7- or 8-chinoxalinyl, 1-, 4-, 5-, 6-, 7- or 8-phthalazinyl. Enclosed are also the respective n-oxides, for example 1-oxy-2-, -3 or -4-pyridyl. Particularly preferred (C₄-C₁₀)heterocyclyl residues are 2- or 3-furyl, 2- or 3-pyrrolyl, 3-, 4- or 5-pyrazolyl, and 2-, 3- or 4-pyridyl. In monosubstituted phenyl groups the substituent can be located in the 2-position, the 3-position or the 4-position, with the 3-position and the 4-position being preferred. If a phenyl group carries two substituents, they can be located in 2,3-position, 2,4-position, 2,5-position, 2,6-position, 3,4-position or 3,5-position. In phenyl groups carrying three substituents the substituents can be located in 2,3,4-position, 2,3,5-position, 2,3,6-position, 2,4,5-position, 2,4,6-position, or 3,4,5-position.

Heteroaryl groups mean an aryl group which comprises, apart from carbon, one or more heteroatoms such as, for example, e.g. 1, 2, 3 or 4 nitrogen atoms, 1 or 2 oxygen atoms, 1 or 2 sulfur atoms or combinations of different hetero atoms, for example, pyridyl, benzothiophene or isoquinolyl.

Halogen means, if not otherwise indicated, fluoro, chloro, bromo or iodo.

The imaging probes of the present invention may be synthesized by using appropriate protecting group chemistry known in the art to build up the central scaffold A and to attach either linker and label this unit via a group L and a group —C(O)—NH—. Appropriate building blocks as well as fluorophores such as the cyanine-dyes (e.g. Cy 3 B, Cy 5.5, Cy 7) are commercially available (e.g. GE-Healthcare). For a subset of probe, described in this invention, the solid-phase synthesis method is particularly useful (B. J. Merrifield, Methods in Enzymology 1997, 289, 3-13). Depending on the synthetic requirements, attachment linker or fluorophore may be done on the solid support or by solution phase chemistry.

In general, reactive side chain residues on the central scaffold A and optionally the group L will be protected and liberated sequentially for further modification with the subunit L1R1. Conjugation of the subunit can be accomplished by known methods of chemical synthesis. Particular useful is the reaction between a dye active ester and a primary amine group of the scaffold A to connect both units via an amide bond. Intermediates as well as final probe molecules may be purified by high performance liquid chromatography (HPLC) and characterized by mass spectrometry and analytical HPLC before they are used in labelling and imaging experiments.

The present invention is illustrated in the following paragraph by several but non-limiting examples:

In a preferred embodiment, the probe of the formula (I) comprises a scaffold A which is derived from a dipeptide cathepsin S inhibitor as shown as compound 62. in Table 2 above and as disclosed in WO2005/082876 bearing a chromophore in the P1 position (variable L1). Chromophores can be fluorescent or non fluorescent. The attachment of such chromophores to the central scaffold is made optionally via linker units.

Preferably, the fluorophore are chosen from the group of xanthene- or cyanine dyes. More preferred are cyanine dyes from the group of carbacyanines, thiacyanines, oxacyanines and azacyanines. Cyanine dyes suitable to be used in the context of the present invention are disclosed in U.S. Pat. No. 5,268,468 and U.S. Pat. No. 5,627,027. They include the dyes with the trademark (Amersham, GE Healthcare) Cy 3, Cy 3B, Cy 3.5, Cy 5, Cy 5.5, Cy 7 and Cy 7.5.

More preferred is a probe of the formula (I) selective for cathepsin S based on a morpholine dipeptide scaffold bearing the dansyl group in the P1 position and a nitrile as warhead (Scheme 3):

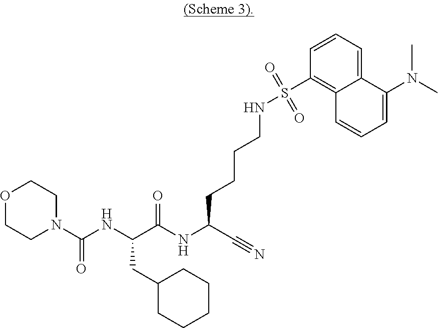

More preferred is a probe of the formula (I) selective for caspase-1 based on a pyridazinodiazepine scaffold bearing the dansyl group in the P4 position and an ethyl acetal as warhead (Scheme 4):

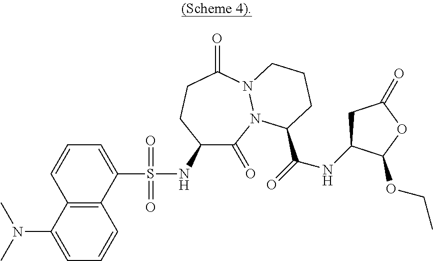

The molecular architecture of compounds of the formula (I) consist of a central scaffold A bearing a group X and a subunit L1R1. Appropriate functional groups for the attachment of subunits L1R1 to scaffold A can be chosen by those skilled in the art, and examples are given below. The specific functional groups L′ in the precursor compound can be placed on the scaffold A for the attachment of suitable L1R1 subunits to yield the group L within the compound of the formula (I) are limited only by the requirement of the synthesis strategy and the final use of such substrate as an activity based imaging reagent. Thus their selection will depend upon the specific reagents chosen to build the desired substrates. Examples of functional groups L′ which can be provided on scaffold A to connect A with the subunit R1L1 include fluoro, chloro, bromo, cyano, nitro, amino, azido, alkylcarbonylamino, carboxy, carbamoyl, alkoxycarbonyl, aryloxycarbonyl, carbaldehyde, hydroxy, alkoxy, aryloxy, alkylcarbonyloxy, arylcarbonyloxy, a carbon-carbon double bond, a carbon-carbon triple bond, and the like. Most preferable examples include amino, azido, hydroxy, cyano, carboxy, carbamoyl, carbaldehyde, or a carbon-carbon double or a carbon-carbon triple bond.

Compounds of the formula L′-A-CO—NH₂ (scaffolds) can be prepared by standard methods known in the art.

The present invention also relates to a method for the preparation of a compound of the formula (I) characterized in

(a) a compound of the formula (II)

L′-A-CO—NH₂ (II)

wherein A is as defined above in its generic and preferred meanings and L′ is fluoro, chloro, bromo, cyano, nitro, amino, azido, alkylcarbonylamino, carboxy, carbamoyl, alkoxycarbonyl, aryloxycarbonyl, carbaldehyde, hydroxy, alkoxy, aryloxy, alkylcarbonyloxy, arylcarbonyloxy, a carbon-carbon double bond, a carbon-carbon triple bond, preferably amino, azido, hydroxy, cyano, carboxy, carbamoyl, carbaldehyde, or a carbon-carbon double or a carbon-carbon triple bond, more preferred amino,

is reacted under conditions known to a skilled person with a compound of the formula L1-R1-H wherein L1 is as defined above in its generic and preferred meanings to a compound of the formula (III)

L1-R1-L-A-CO—NH₂ (III)

(b) the compound (III) is reacted with C₃N₃Cl₃ to a compound of the formula (I) with a nitrile group as warhead.

Preferably cysteine protease substrates functionalized with a label are synthesized on the solid support. Depending on the compatibility of the label for solid phase synthesis a combination of solid-support and solution-phase synthesis is used.

The preparation of a compound of the formula (I) wherein group A consists of a cathepsin S inhibitor, L1 is a dansyl group is further described in Example 1: The scaffold of Example 1 having a C-terminal lysine residue functionalized with the dansyl group in the side chain is prepared on the solid-support using the sieber amide resin. The obtained C-terminal amide is converted into the nitrile by treating with cyanuric chloride (C₃N₃Cl₃). The final product was purified by preparative HPLC.

Compounds of the formula L′-A-CO₂H (scaffolds) can be prepared by standard methods known in the art.

The present invention also relates to a method for the preparation of a compound of the formula (I) characterized in

(a) a compound of the formula (IV)

L′-A-CO₂H (IV)

wherein A is as defined above in its generic and preferred meanings and L′ is fluoro, chloro, bromo, cyano, nitro, amino, azido, alkylcarbonylamino, carboxy, carbamoyl, alkoxycarbonyl, aryloxycarbonyl, carbaldehyde, hydroxy, alkoxy, aryloxy, alkylcarbonyloxy, arylcarbonyloxy, a carbon-carbon double bond, a carbon-carbon triple bond, preferably amino, azido, hydroxy, cyano, carboxy, carbamoyl, carbaldehyde, or a carbon-carbon double or a carbon-carbon triple bond, more preferred amino,

is reacted under conditions known to a skilled person with a compound of the formula L1-R1-H wherein L1 is as defined above in its generic and preferred meanings to a compound of the formula (V)

L1-R1-L-A-CO₂H (V)

(b) the compound (V) is reacted with a warhead X to a compound of the formula (I).

Preferably protease substrates functionalized with a label are synthesized on the solid support. Depending on the compatibility of the label for solid phase synthesis a combination of solid-support and solution-phase synthesis is used.

For the synthesis of several inhibitors with a peptidomimetic structure non-peptidic building blocks may be utilized for the solid-phase synthesis.

Building block (VI) is preferably used for the synthesis of caspase-1 probes, e.g. the compounds of Examples 3 and 4.

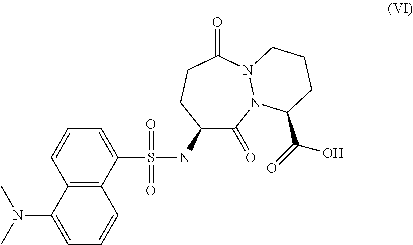

Building block (VII) is preferably used for the synthesis of caspase-1 probes, e.g. the compounds of Example 4.

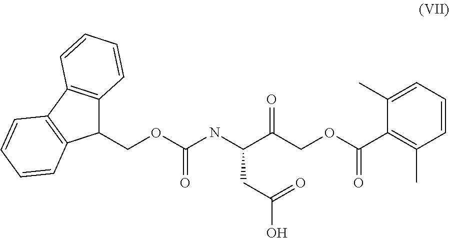

The probes of the present inventions are preferably probes for cathepsin K, cathepsin S, cathepsin B, caspase-1, caspase-3, caspase-8, MMP-13 and TAFI.

The probes of the present invention are used in the context of molecular imaging in vitro, in cell-culture experiments, ex-vivo experiments or in a living organism (in vivo), including screening and whole animal imaging. Mostly preferred are imaging modalities such as optical imaging and magnetic resonance imaging (MRI).

The probes of the present invention are intended to be used for diagnostic imaging of protease activity. Most preferred are applications which provide methods of monitoring the effect of a drug or drug-like substance towards the targeted proteases. Administration of such a drug or drug like substance should have a measurable effect to the signal from the probe of the present invention.

A further most preferred aspect of the probes of the present invention is their use as imaging reagents in surgical guidance and to monitor the effect of medical treatment. Surgical guidance includes the detection of tumours margin and detection of progression of tumours metastasis.

Therefore, a further aspect of the present invention is method of imaging a living organism, comprising:

a) administering to said organism a probe of the formula (I),

(b) exposing said organism to electromagnetic radiation which excites fluorophore to produce a detectible signal; and

(c) detecting said signal and creating an image thereby.

A “living organism” may be any live cell or whole organism comprising the cysteine protease to-be-detected, preferably the living organism is a mammal, e.g. a mouse or a rat.

The probes of the present invention are highly selective, whereby a risk of false positives can be avoided.

Abbreviations:

Boc=N-tert-Butyloxycarbonyl

DMF=dimethylformamide

DMSO=dimethylsulfoxide

DCM=dichloromethane

equiv.=equivalents

sat.=saturated

THF=tetrahydrofuran

DIC=N,N′-diisopropylcarbodiimide

DIPEA=diisopropyl-ethyl amine

HOAt=1-Hydroxy-7-azabenzotriazole

HOBt=1-hydroxybenzotriazol

HATU=O-7-Azabenzotriazol-1-yl-N,N,N′,N′-tetramethyl-uronium-hexafluoro-phosphate

HBTU=O-benzotriazole-N,N,N′,N′-tetramethyl-uronium-hexafluoro-phosphate

NHS=N-hydroxysuccinimidyl ester

FMOC=9-fluoromethyl-chloroformate

OSu=succinimidyl ester

Generally, the probes may be synthesized using standard protocols for solid phase peptide synthesis.

General Procedure for Solid Phase Peptide Synthesis on Sieber Resin:

For the loading of the Sieber resin, the resin was treated two times for 15 minutes with 30% piperidine/DMF solution. The resin was washed with DCM and DMF. 4 equiv. of FMOC-protected amino acid, 4 equiv. of HBTU, 4 equiv. of HOBt and 8 equiv. of DIPEA were solved in DCM/DMF (1/1) and the reaction mixture was added to the resin (loading: 0.72 mmol/g). The reaction mixture was stirred at room temperature over night. The resin was washed with DCM and DMF. For FMOC-deprotection the resin was treated two times for 15 minutes with 30% piperidine/DMF solution. For solid phase peptide synthesis a standard protocol was used: 4 equiv. of FMOC-protected amino acid, 4 equiv. of HBTU, 4 equiv. of HOBt and 8 equiv. of DIPEA were solved in a mixture of DCM/DMF (1/1). The reaction mixture is shaken at room temperature for 20 minutes and then added to the resin. The reaction mixture was incubated for 2 hours. For cleavage from the solid phase, the resin was treated with 50% TEA in DCM. The solvent was co-evaporated with toluene under reduced pressure and the final product was purified by preparative HPLC (Gradient: H₂O+0.05% TEA; 5 to 95% CH₃CN).

Obtained amides were converted into the nitriles using a standard protocol: The amide was solved in DMF. At 0° C. 0.65 equiv. of cyanuric chloride (C₃N₃Cl₃) was given in one portion to the reaction mixture. The reaction mixture was stirred over night at room temperature. Ice water was added to the reaction which was then extracted with DCM. The combined organic phases were washed with brine and dried over MgSO₄. The solvent was removed under reduced pressure and the final product was purified by chromatography on silica gel or preparative HPLC.

General Procedure for Solid Phase Peptide Synthesis on 2-Chlorotrityl-Resin:

For the loading of the 2-chlorotrityl-resin, 2 equiv. of FMOC-protected amino acid and 3 equiv. of DIPEA were solved in DCM and the reaction mixture was added to the resin (loading: 1.4 mmol/g). The reaction mixture was shaken at room temperature over night. The resin was washed with DCM and DMF. For FMOC-deprotection the resin was treated two times for 15 minutes with 30% piperidine/DMF solution. For solid phase peptide synthesis a standard protocol was used: 4 equiv. of acid, 4 equiv. of HATU, 4 equiv. of HOAt and 8 equiv. of DIPEA were solved in a mixture of DCM/DMF (1/1). The reaction mixture was stirred at room temperature for 20 minutes and then added to the resin. The reaction mixture was shaken for 2 hours or longer if the FMOC-protected amino acid were sterically hindered. For cleavage from the solid phase, the resin was treated with 5% TEA in DCM two times for 15 minutes. The solvent was coevaporated with toluene under reduced pressure and the final product was purified by preparative HPLC (Gradient: H₂O+0.05% TEA; 5 to 95% CH₃CN).

General Procedure for Solid Phase Peptide Synthesis on Aminomethylpolystyrene Resin:

Aminomethylpolystyrene resin was modified with a carbazate linker according to the procedure described in D. Kato et al., Nat. Chem. Biol. 2005, 1, 33-38. For the loading of the modified aminomethylpolystyrene resin 2 equiv. of FMOC-protected amino acyloxymethyl ketone was solved in DMF and the reaction mixture was added to the resin (loading: 1.4 mmol/g). The reaction mixture was shaken at 50° C. over night. The resin was washed with DCM and DMF. For FMOC-deprotection the resin was treated two times for 30 minutes with 7% NHEt₂/DMF solution. For solid phase peptide synthesis a standard protocol was used: 3 equiv. of acid, 3 equiv. of HOBt and 3 equiv. of DIC were solved in a mixture of DMF. The reaction mixture was stirred at room temperature for 20 minutes and then added to the resin. The reaction mixture was shaken for 4 hours or longer if the FMOC-protected amino acid was sterically hindered. For cleavage from the solid phase, the resin was treated with 5% H₂O in TEA for 1.5 hours. The solvent was coevaporated with toluene under reduced pressure and the final product was purified by preparative HPLC (Gradient: H₂O+0.05% TEA; 5 to 95% CH₃CN).

### Example 1

### Cathepsin S Probe

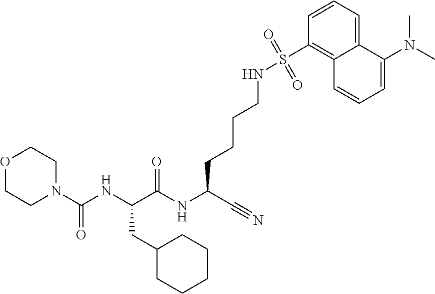

The compound was prepared according to the procedure for Solid Phase Peptide Synthesis on Sieber resin, and purified by HPLC (H₂O+0.05% TEA; 4-95% CH₃CN). Calculated: [M+H]⁺=627.8, found: [M+H]⁺=627.2. Yield: 72%.

### Example 2

### Cathepsin K Probe

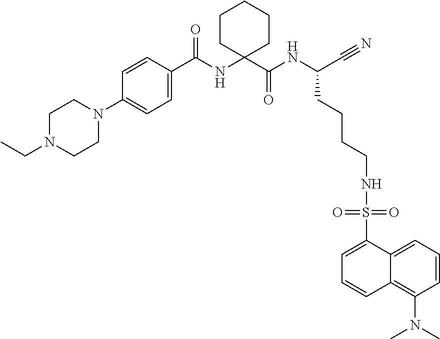

The compound was prepared according to the procedure for Solid Phase Peptide Synthesis on Sieber resin, and purified by HPLC (H₂O+0.05°% TEA; 4-95% CH₃CN). Calculated: [M+H]⁺=702.9, found: [M+H]⁺=702.3. Yield: 72%.

### Example 3

### Caspase-1 Probe

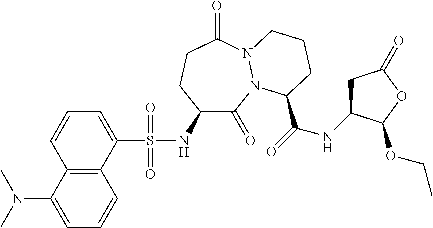

70.4 mg (0.3 mmol) of ((2R,3S)-2-Ethoxy-5-oxo-tetrahydro-furan-3-yl)-carbamic acid allyl ester (WO9903852) was dissolved in 5 ml DCM. 48 mg (0.3 mmol) 1,3-Dimethylbarbituric acid and 29.5 mg (0.0025 mmol) tetrakistriphenylphosphine Palladium (0) were added in portions. The solution turned red after a minute. After 1 h, 121 mg (0.25 mmol) of Building block (VI), 107 mg HATU, 38 mg HOAt and 80 μl DIPEA in 5 ml DCM were added. The reaction was stirred at room temperature for 12 h. The reaction mixture was washed with water, 0.5 M NaHSO₄-solution and brine. The organic phase was concentrated and the product purified by silica gel column chromatography (DCM/MeOH) to yield 0.068 g. Calculated: [M+H]⁺=601.6, found: [M+H]⁺=602.

### Example 4

### Caspase-1 Probe

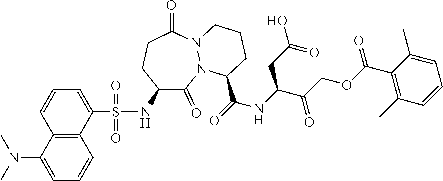

The compound was prepared according to the procedure for Solid Phase Peptide Synthesis on 2-chlorotrityl-resin, and purified by HPLC (H₂O+0.05% TEA; 4-95% CH₃CN). Calculated: [M+H]⁺=735.8, found: [M+H]⁺=736.2. Yield: 11%.

### Example 5

### Building Block for Caspase-1 Probe of Example 3 and 4

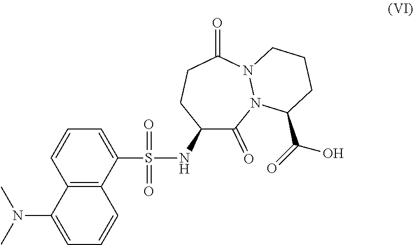

Building block (VI) has been prepared in two steps.

### Step 1: (1S,9S)-9-(5-Dimethylamino-naphthalene-1-sulfonylamino)-6,10-dioxo-octahydro-pyridazino[1,2-a][1,2]diazepine-1-carboxylic acid methyl ester

1 g (3.7 mmol) Dansylchloride was dissolved in 10 ml dry DMF and a solution of 946 mg (3.7 mmol) of (1S,9S)-9-Amino-6,10-dioxo-octahydro-pyridazino[1,2-a][1,2]diazepine-1-carboxylic acid methyl ester (WO01047930) and 0.647 ml DIPEA in 5 ml dry DMF were added dropwise. The reaction mixture was stirred at room temperature over night. The solvent was evaporated and the crude product purified by silica gel column chromatography (DCM:MeOH, 50:1 to 10:1) to yield 1.4 g of a slightly brown solid.

### Step 2: (1S,9S)-9-(5-Dimethylamino-naphthalene-1-sulfonylamino)-6,10-dioxo-octahydro-pyridazino[1,2-a][1,2]diazepine-1-carboxylic acid

1.0 g (2 mmol) of (1S,9S)-9-(5-Dimethylamino-naphthalene-1-sulfonylamino)-6,10-dioxo-octahydro-pyridazino[1,2-a][1,2]diazepine-1-carboxylic acid methyl ester was dissolved in THF/H₂O (3:1) and cooled to 0° C. To this solution, 0.12 g (5.2 mmol) LiOH was added and the reaction mixture was stirred at room temperature over night. The reaction mixture was acidified with HCl (0.5N) and extracted with ethylacetate. The combined organic phases were washed with H₂O, dried over MgSO₄ and the solvent evaporated in vacuo. The compound was purified on a silica gel column chromatography (DCM/MeOH) to yield 0.9 g of a yellow solid.

### Example 6

### Building Block for Caspase-1 Probe of Example 4

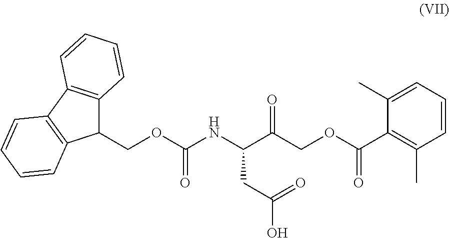

Building block (VII) has been prepared according to the procedure described in D. Kato et al., Nat. Chem. Biol. 2005, 1, 33-38.

### Example 7

### Cathepsin-S Probe

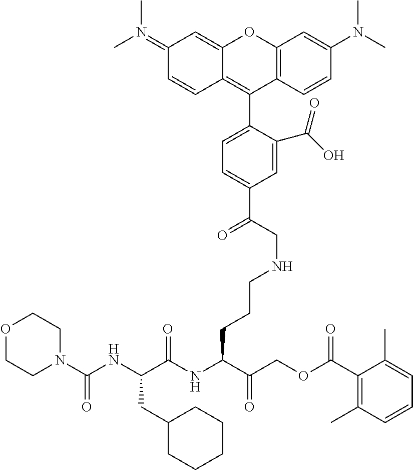

Boc-group of building block (VIII) of Example 10 was removed by treatment with a 50% TFA/CH₂Cl₂ solution for 10 minutes at room temperature. The solvent was coevaporated with toluene and the residue was solved in DMF. 1 equiv. of Tetramethylrhodamine-OSu and 6 equiv. of DIPEA were added to the reaction mixture. The reaction mixture was stirred at room temperature for 12 h. The solvent was removed and the final product was purified by preparative HPLC (H₂O+0.05% TEA; 4-95% CH₃CN). Calculated: [M+H]⁺=973.2, found: [M+H]⁺=972.6. Yield: 66%.

### Example 8

### Cathepsin-S Probe

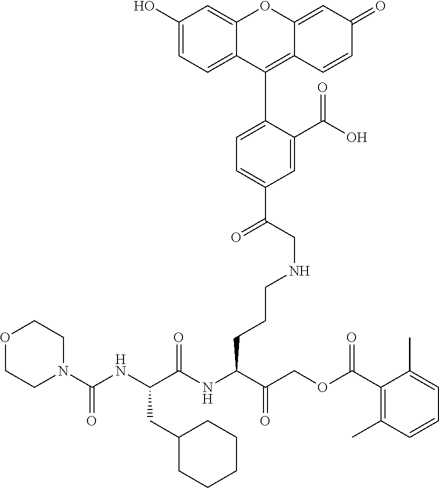

Boc-group of building block (VIII) of Example 10 was removed by treatment with a 50% TFA/CH₂Cl₂ solution for 10 minutes at room temperature. The solvent was coevaporated with toluene and the residue was solved in DMF.1 equiv. of Fluoresceine-OSu and 6 equiv. of DIPEA were added to the reaction mixture. The reaction mixture was stirred at room temperature for 12 h. The solvent was removed and the final product was purified by preparative HPLC (H₂O+0.05% TEA; 4-95% CH₃CN). Calculated: [M+H]⁺=917.0, found: [M+H]⁺=917.5. Yield: 73%.

### Example 9

### Cathepsin-S Probe

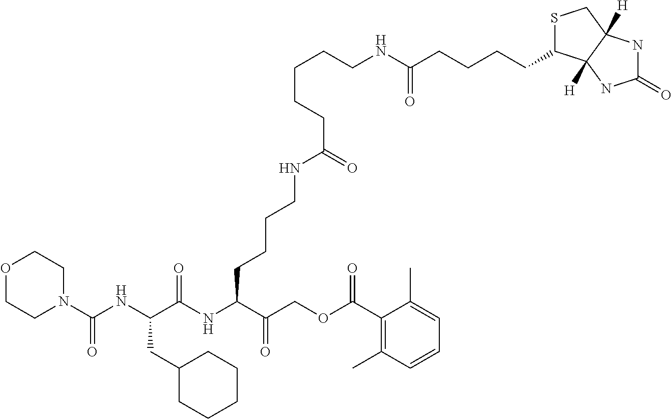

Boc-group of building block (VIII) of Example 10 was removed by treatment with a 50% TFA/CH₂Cl₂ solution for 10 minutes at room temperature. The solvent was coevaporated with toluene and the residue was solved in DMF.1 equiv. of sulfosuccinimidyl-6-(biotinamido)hexanoate and 6 equiv. of DIPEA were added to the reaction mixture. The reaction mixture was stirred at room temperature for 12 h. The solvent was removed and the final product was purified by preparative HPLC (H₂O+0.05% TEA; 4-95% CH₃CN). Calculated: [M+H]⁺=898.2, found: [M+H]⁺=897.9. Yield: 62%.

### Example 10

### Building Block for Cathepsin-S Probe of Example 7, 8 and 9

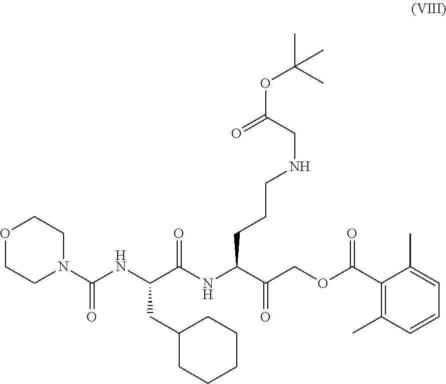

Building block (VIII) was prepared according to the procedure for Solid Phase Peptide Synthesis on aminomethylpolystyrene resin, and purified by HPLC (H₂O+0.05% TEA; 4-95% CH₃CN). Calculated: [M+H]⁺=558.7, found: [M+H]⁺=558.2. Yield: 32%.

### Example 11

### Building Block for the Preparation of Building Block (VIII) of Example 10

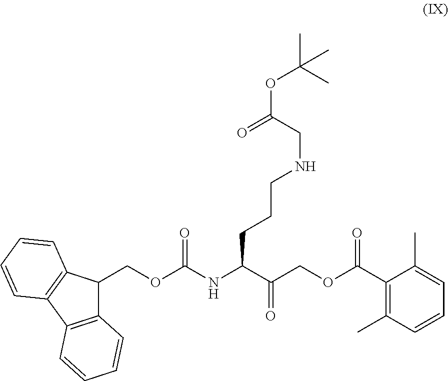

Building block (IX) was prepared according to the procedure described in D. Kato et al., Nat. Chem. Biol. 2005, 1, 33-38.

### Example 12

### Cathepsin-B Probe

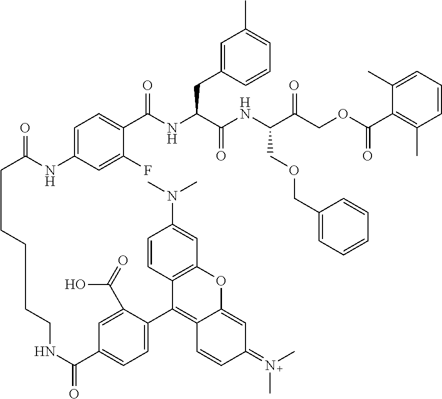

Fmoc-group of building block (X) of Example 15 was removed by treatment with a 20% NHEt₂/CH₂Cl₂ solution for 10 minutes at room temperature. The solvent was coevaporated with toluene and the residue was solved in DMF.1 equiv. of Tetramethylrhodamine-OSu and 6 equiv. of DIPEA were added to the reaction mixture. The reaction mixture was stirred at room temperature for 12 h. The solvent was removed and the final product was purified by preparative HPLC (H₂O+0.05% TEA; 4-95% CH₃CN). Calculated: [M+Na]⁺=1167.3, found: [M+Na]⁺=1167.7. Yield: 84%.

### Example 13

### Cathepsin-B Probe

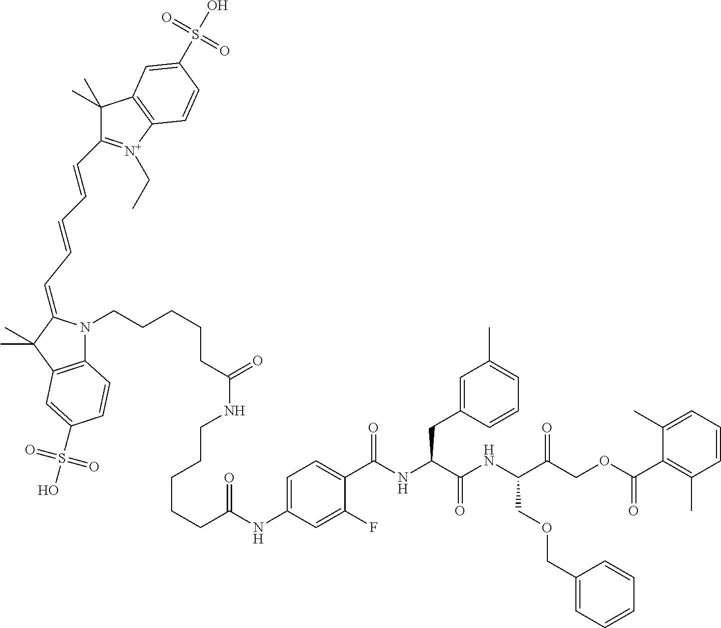

Fmoc-group of building block (X) of Example 15 was removed by treatment with a 20% NHEt₂/CH₂Cl₂ solution for 10 minutes at room temperature. The solvent was coevaporated with toluene and the residue was solved in DMF.1 equiv. of Cy5-OSu and 6 equiv. of DIPEA were added to the reaction mixture. The reaction mixture was stirred at room temperature for 12 h. The solvent was removed and the final product was purified by preparative HPLC (H₂O+0.05% TEA; 4-95% CH₃CN). Calculated: [M+Na]⁺=1392.7, found: [M+Na]⁺=1392.6. Yield: 78%.

### Example 14

### Cathepsin-B Probe

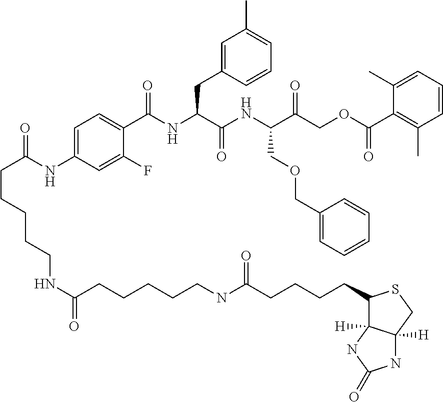

Fmoc-group of building block (X) of Example 15 was removed by treatment with a 20% NHEt₂/CH₂Cl₂ solution for 10 minutes at room temperature. The solvent was coevaporated with toluene and the residue was solved in DMF.1 equiv. of sulfosuccinimidyl-6-(biotinamido)hexanoate and 6 equiv. of DIPEA were added to the reaction mixture. The reaction mixture was stirred at room temperature for 12 h. The solvent was removed and the final product was purified by preparative HPLC (H₂O+0.05% TEA; 4-95% CH₃CN). Calculated: [M+Na]⁺=1092.3, found: [M+Na]⁺=1092.7. Yield: 35%.

### Example 15

### Building Block for Cathepsin-B Probe of Example 12, 13 and 14

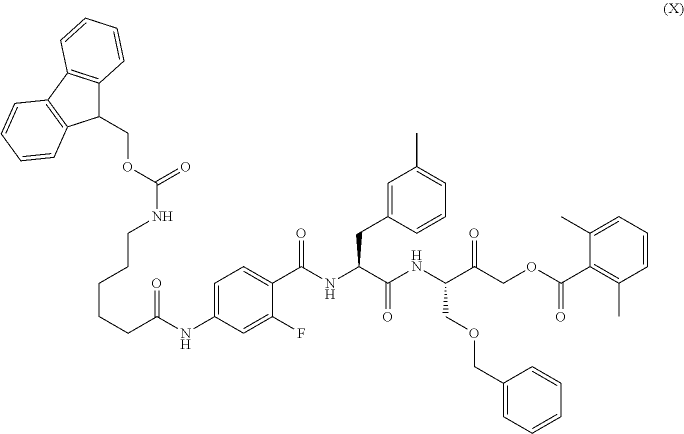

Building block (X) was prepared according to the procedure for Solid Phase Peptide Synthesis on aminomethylpolystyrene resin, and purified by HPLC (H₂O+0.05% TEA; 4-95% CH₃CN). Calculated: [M+H]⁺=975.1, found: [M+H]⁺=975.5. Yield: 30%.

### Example 16

### Building Block for the Preparation of Building Block (X) of Example 15

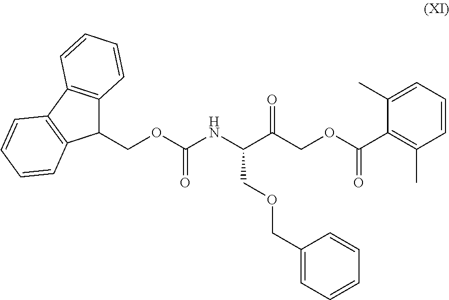

Building block (XI) was prepared from N-α-Fmoc-O-benzyl-L-serine according to the procedure described in D. Kato et al., Nat. Chem. Biol. 2005, 1, 33-38.

What is claimed is:

1. A molecular probe for proteases of the formula (I)
L1R1-L-A-X (I)
wherein
X is an electrophilic warhead; or X is a hydrogen;
A is a group recognizable by a protease;
R1 is a linker;
L is a bond or a group allowing for a facile conjugation of the group R1, and
L1 is a label optionally bound to a solid support.

2. The molecular probe according to claim 1, wherein the protease is a cysteine protease from the cathepsin or caspase subfamily, or a metalloprotease from the MMP or carboxypeptidase subfamily.

3. The molecular probe according to claim 2, wherein the cysteine protease from the cathepsin or caspase subfamily is selected from cathepsin K, cathepsin S, cathepsin B, caspase-1, caspase-3 or caspase-8, and the metalloprotease from the MMP or carboxypeptidase subfamily is selected from MMP-13 or TAFI.

4. The molecular probe according to claim 1, wherein L is a direct bond or a group selected from 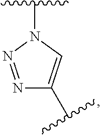
—(NRx)-, —O—, —C═N—, —C(═O)—, —C(═O)—NH—, —NH—C(═O)—, —C(═O)H, —CRx=CRy-, —C≡C— and phenyl, wherein Rx and Ry are independently H or (C₁-C₆)alkyl.

5. The molecular probe according to claim 1, wherein
R1 is a straight or branched chain alkylene group with 1 to 300 carbon atoms, wherein optionally
(a) one or more carbon atoms are replaced by oxygen;
(b) one or more carbon atoms are replaced by nitrogen carrying a hydrogen atom, and the adjacent carbon atoms are substituted by oxo, representing an amide function —NH—CO—;
(c) one or more carbon atoms are replaced by an ester function —O—CO—;
(d) the bond between two adjacent carbon atoms is a double or a triple bond; or
(e) two adjacent carbon atoms are replaced by a disulfide linkage,
or a combination thereof.

6. The molecular probe according to claim 5, wherein when
(a) one or more carbon atoms are replaced by oxygen, then every third carbon atom is replaced by oxygen.

7. The molecular probe according to claim 6, wherein R1 is a polyethyleneoxy group with 1 to 100 ethyleneoxy units;

8. The molecular probe according to claim 1, wherein R1 is alkyl or a straight or branched chain alkylene group with 1 to 50 carbon atoms, wherein one or more carbon atoms are replaced by oxygen.

9. The molecular probe according to claim 8, wherein every third carbon atom is replaced by oxygen.

10. The molecular probe according to claim 9, wherein R1 is a polyethyleneoxy group with 1 to 20 ethyleneoxy units.

11. The molecular probe according to claim 1, wherein L1 is a spectroscopic probe; a chromophore; a magnetic probe; a contrast reagent; a molecule which is one part of a specific binding pair which is capable of specifically binding to a partner; a molecule covalently attached to a solid support, where the support may be a glass slide, a microtiter plate or any polymer known to those proficient in the art; a biomolecule with desirable enzymatic, chemical or physical properties; or a molecule possessing a combination of any of the properties listed above; or a positively charged linear or branched polymer.

12. The molecular probe according to claim 11, wherein L1 is a spectroscopic probe which is a fluorophore

13. The molecular probe according to claim 1, wherein X is an electrophilic warhead.

14. The molecular probe according to claim 13, wherein X is a group selected from 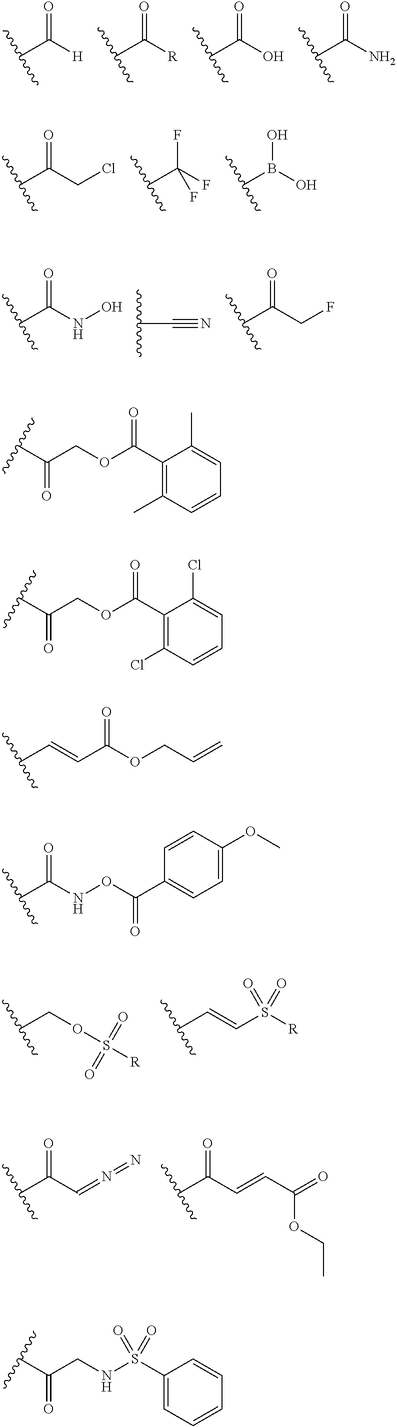 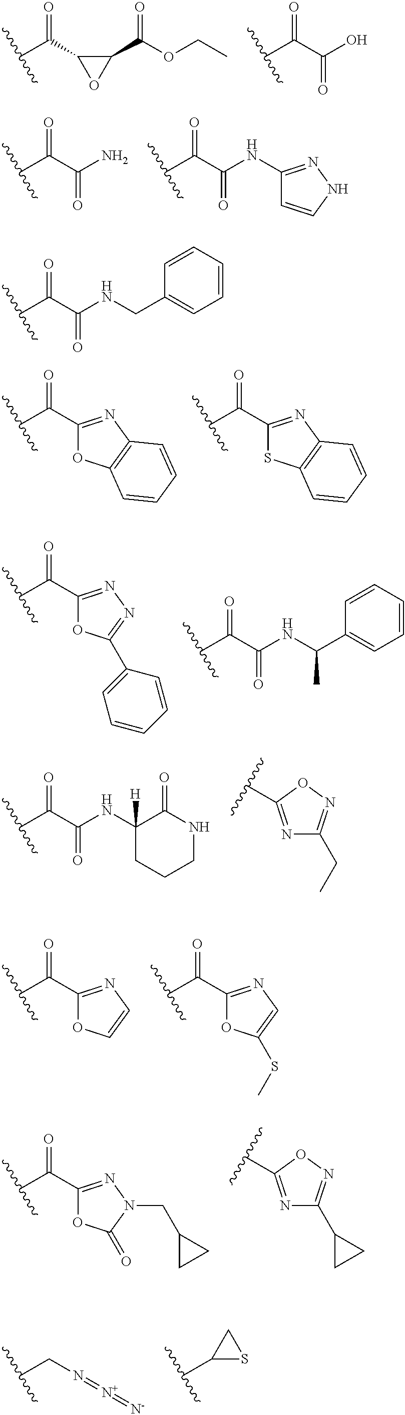 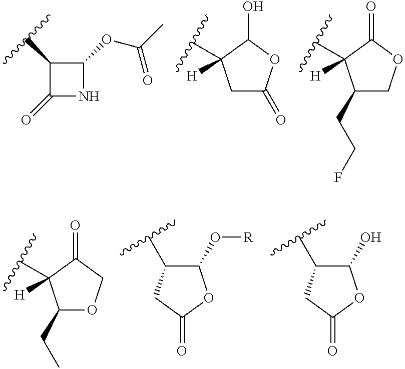
wherein R=alkyl, aryl.

15. The molecular probe according to claim 1, selected from one of the compounds 1.-61.:

| | |
|---|---|
| 1. | 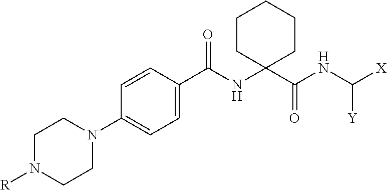 |
| 2. | 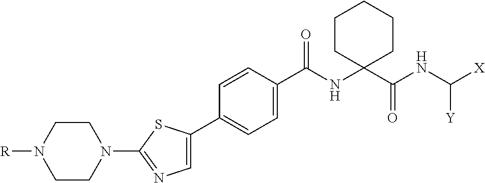 |
| 3. | 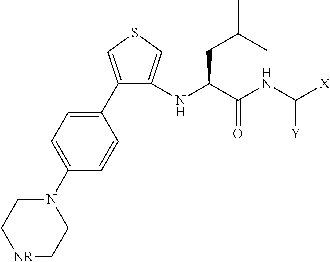 |
| 4. | 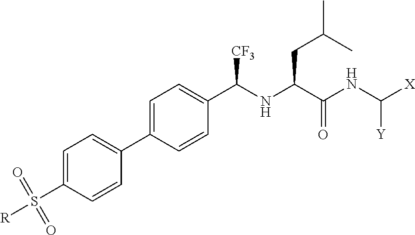 |
| 5. | 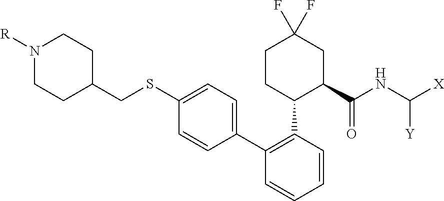 |
| 6. | 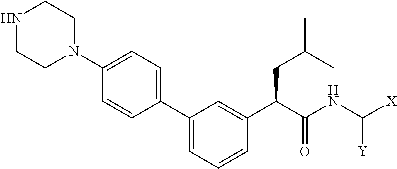 |
| 7. | 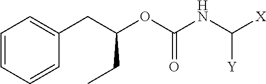 |
| 8. | 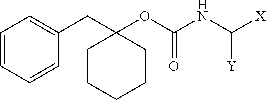 |
| 9. | 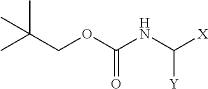 |
| 10. | 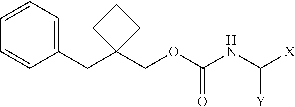 |
| 11. | 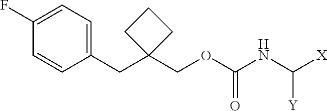 |
| 12. | 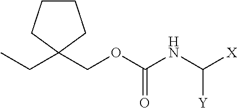 |
| 13. | 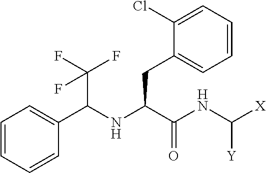 |
| 14. | 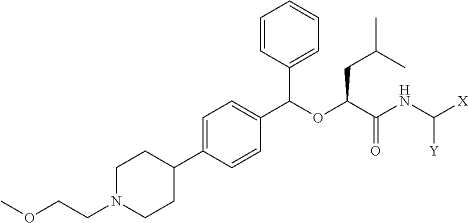 |
| 15. | 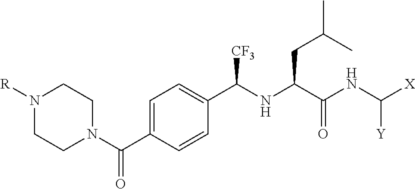 |
| 16. | 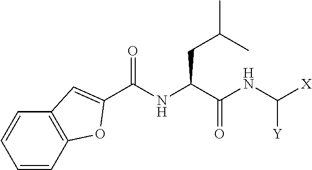 |
| 17. | 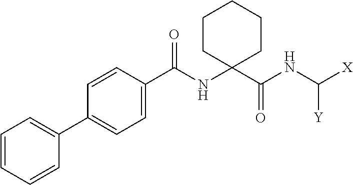 |
| 18. | 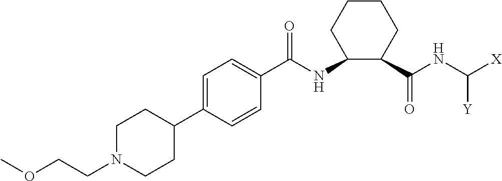 |
| 19. | 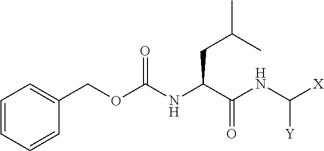 |
| 20. | 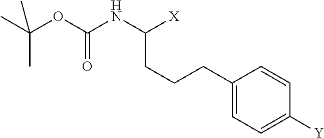 |
| 21. | 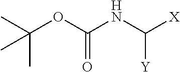 |
| 22. | 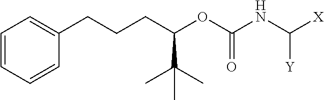 |
| 23. | 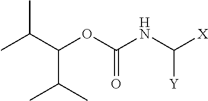 |
| 24. | 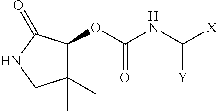 |
| 25. | 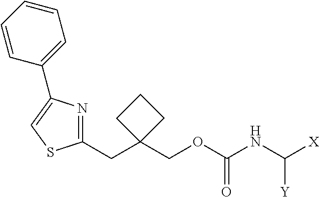 |
| 26. | 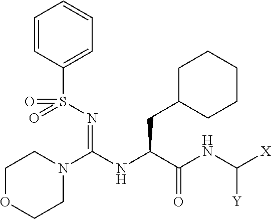 |
| 27. | 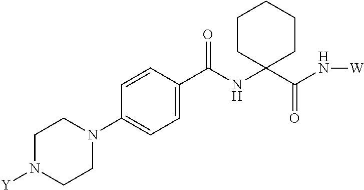 |
| 28. | 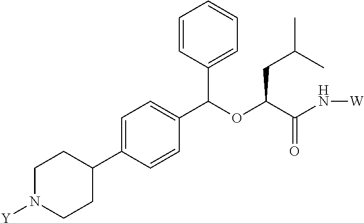 |
| 29. | 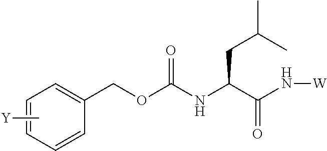 |
| 30. | 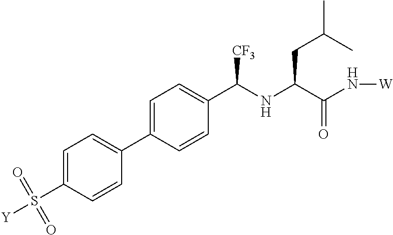 |
| 31. | 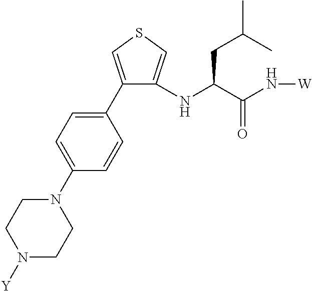 |
| 32. | 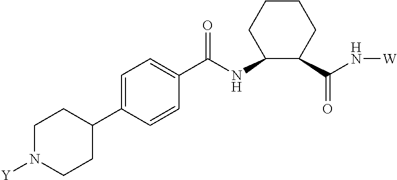 |
| 33. | 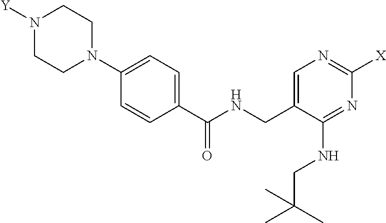 |
| 34. | 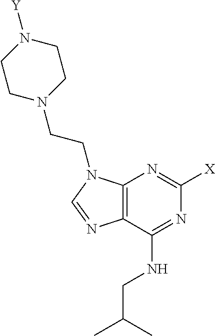 |
| 35. | 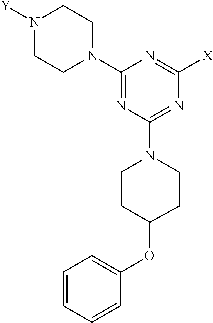 |
| 36. | 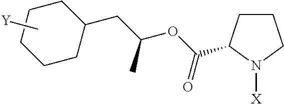 |
| 37. | 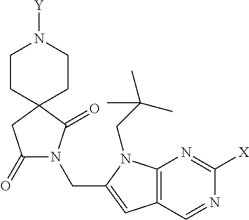 |
| 38. | 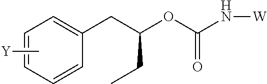 |
| 39. | 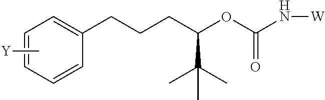 |
| 40. | 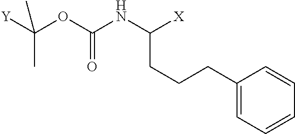 |
| 41. | 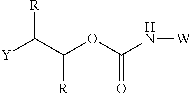 |
| 42. | 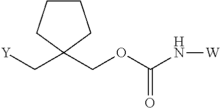 |
| 43. | 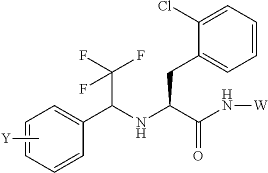 |
| 44. | 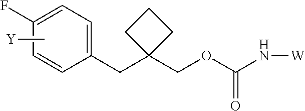 |
| 45. | 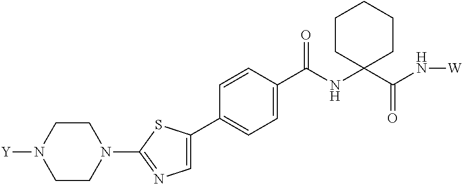 |
| 46. | 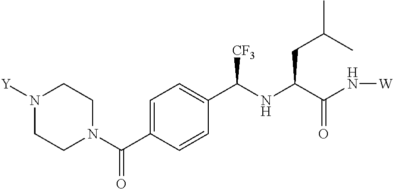 |
| 47. | 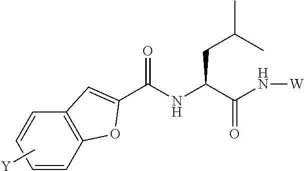 |
| 48. | 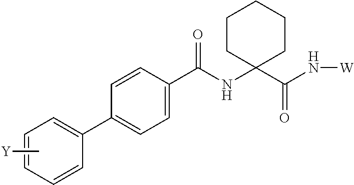 |
| 49. | 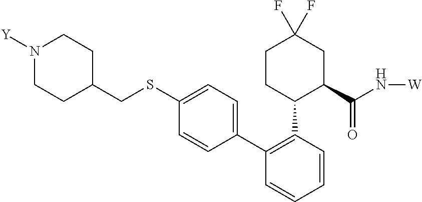 |
| 50. | 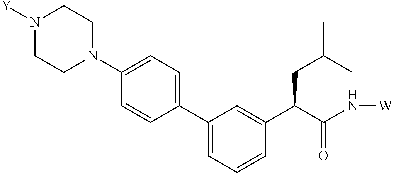 |
| 51. | 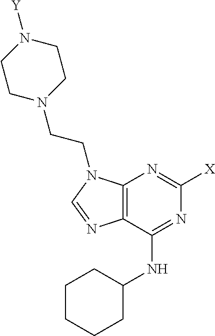 |
| 52. | 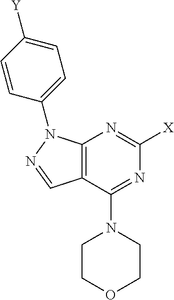 |
| 53. | 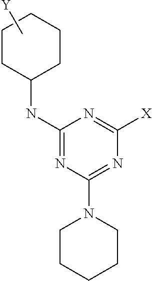 |
| 54. | 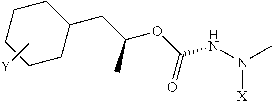 |
| 55. | 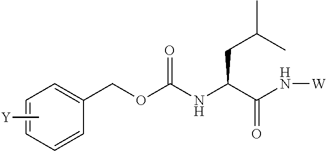 |
| 56. | 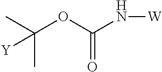 |
| 57. | 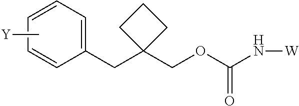 |
| 58. | 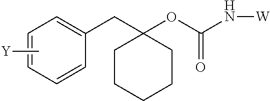 |
| 59. | 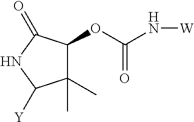 |
| 60. | 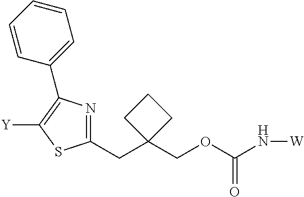 |
| 61. | 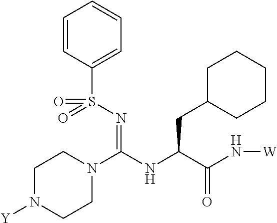 |

wherein independently of each other the variables in the compounds 1. to 61. are defined as indicated in the definition next to the respective compound; Y is -L-R1-L1; and R1, L and L1 are as described in claim 1; R is H or (C₁-C₆)-alkyl; 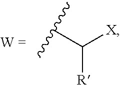
or W═X,
wherein X is defined as a group selected from 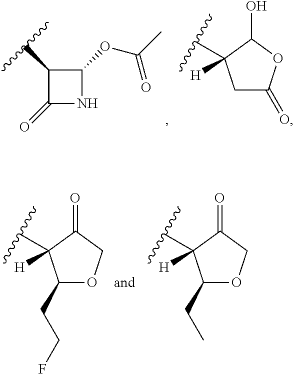
and R′ is H; or C₁-C₆-alkyl optionally substituted by
OH, —O—C₁-C₆-alkyl, —O—(C═O)—O—C₁-C₆-alkyl, SH, —S—C₁-C₆-alkyl, NH₂, —NH—C₁-C₆-alkyl, —N(C₁-C₆-alkyl)₂, —(C═O)—NH—C₁-C₆-alkyl, —COOH, —C(═O)O—C₁-C₆-alkyl or —NH—C(C═O)—C₁-C₆-alkyl; or aryl.

16. The molecular probe according to claim 15 wherein the aryl optional substituent group is a phenyl.

17. The molecular probe according to claim 1, selected from one of the compounds 62.-114.:

| | |
|---|---|
| 62. | 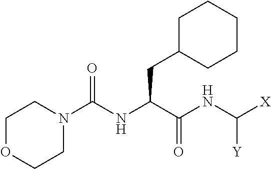 |
| 63. | 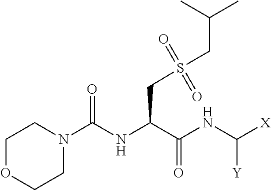 |
| 64. | 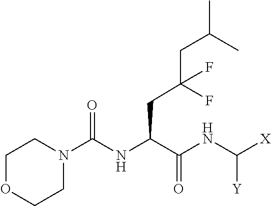 |
| 65. | 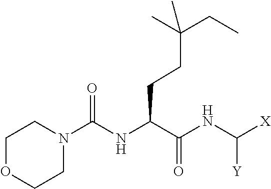 |
| 66. | 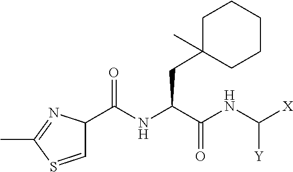 |
| 67. | 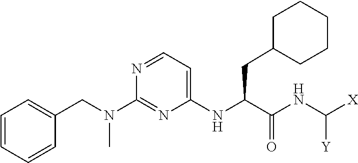 |
| 68. | 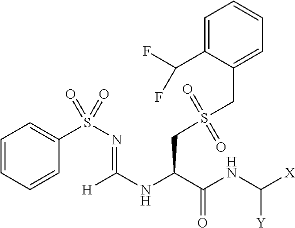 |
| 69. | 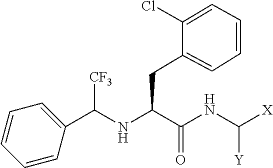 |
| 70. | 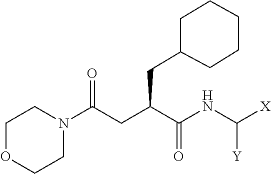 |
| 71. | 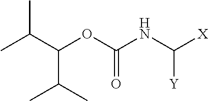 |
| 72. | 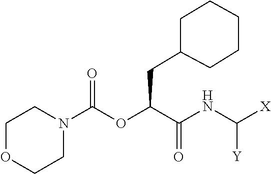 |
| 73. | 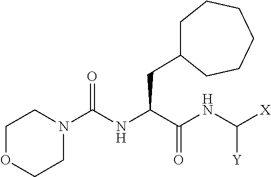 |
| 74. | 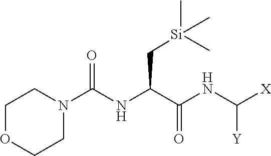 |
| 75. | 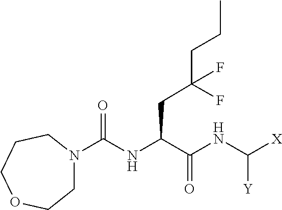 |
| 76. | 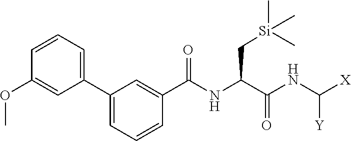 |
| 77. | 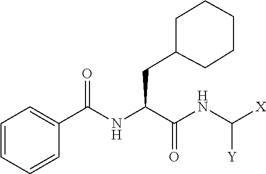 |
| 78. | 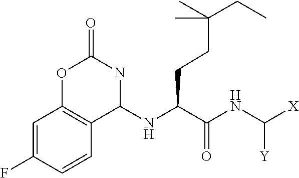 |
| 79. | 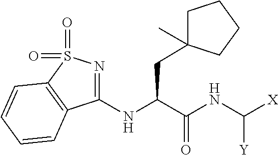 |
| 80. | 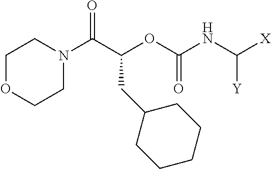 |
| 81. | 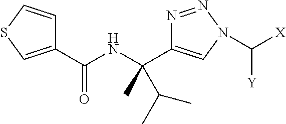 |
| 82. | 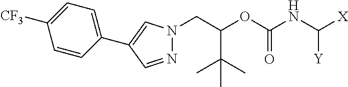 |
| 83. | 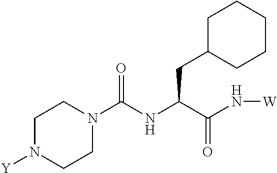 |
| 84. | 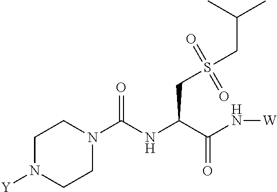 |
| 85. | 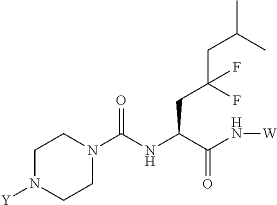 |
| 86. | 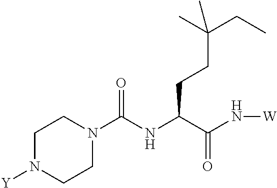 |
| 87. | 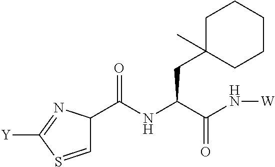 |
| 88. | 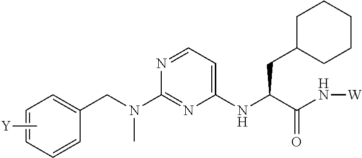 |
| 89. | 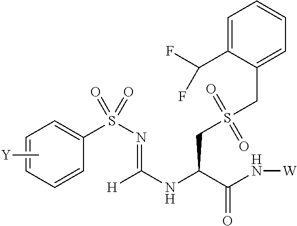 |
| 90. | 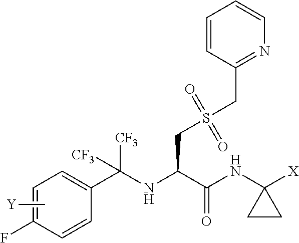 |
| 91. | 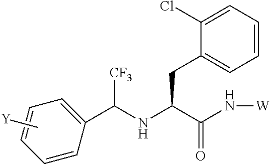 |
| 92. | 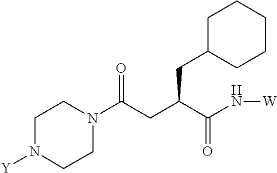 |
| 93. | 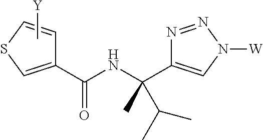 |
| 94. | 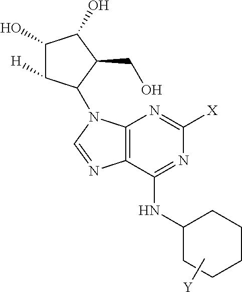 |
| 95. | 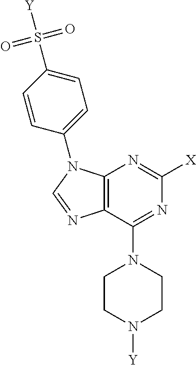 |
| 96. | 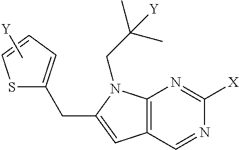 |
| 97. | 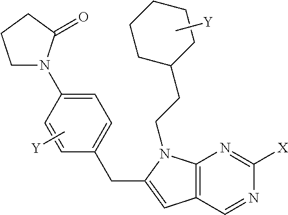 |
| 98. | 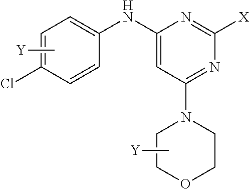 |
| 99. | 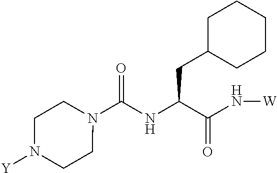 |
| 100. | 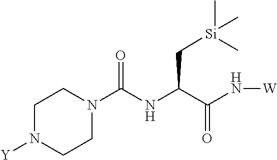 |
| 101. | 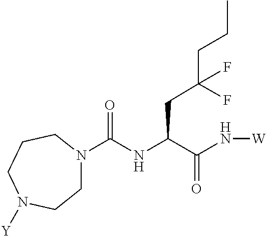 |
| 102. | 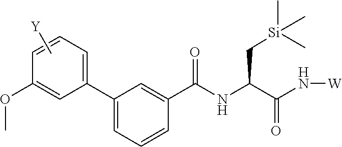 |
| 103. | 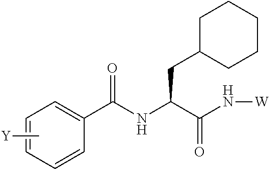 |
| 104. | 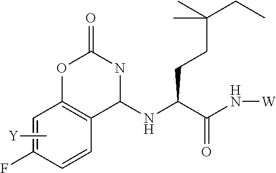 |
| 105. | 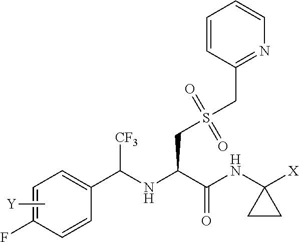 |
| 106. | 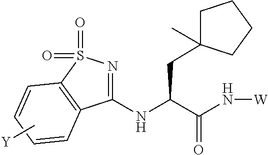 |
| 107. | 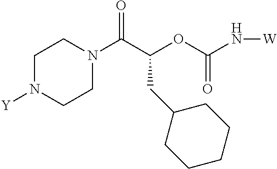 |
| 108. | 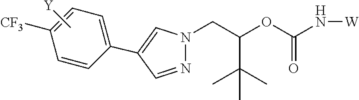 |
| 109. | 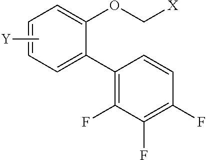 |
| 110. | 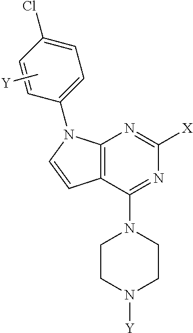 |
| 111. | 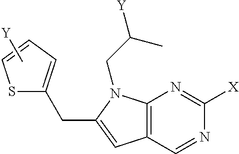 |
| 112. | 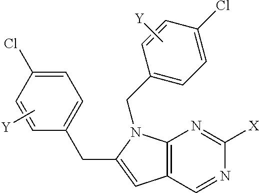 |
| 113. | 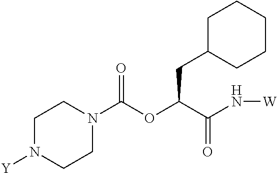 |
| 114. | 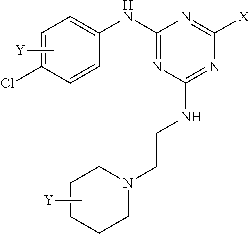 |

wherein independently of each other the variables in the compounds 62. to 114. are defined as indicated in the definition next to the respective compound; Y is -L-R1-L1; and R1, L and L1 are as described in claim 1; 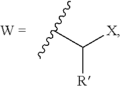
or W═X,
wherein X is defined as a group selected from 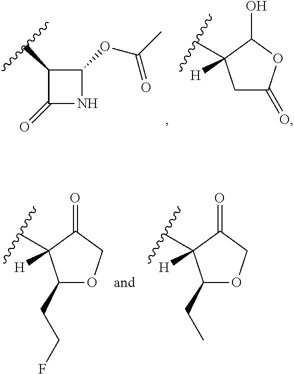
and R′ is H; or C₁-C₆-alkyl optionally substituted by
OH, —O—C₁-C₆-alkyl, —O—(C═O)—O—C₁-C₆-alkyl, SH, —S—C₁-C₆-alkyl, NH₂, —NH—C₁-C₆-alkyl, —N(C₁-C₆-alkyl)₂, —(C═O)—NH—C₁-C₆-alkyl, —COOH, —C(═O)O—C₁-C₆-alkyl or —NH—C(C═O)—C₁-C₆-alkyl; or aryl.

18. The molecular probe according to claim 17 wherein the aryl optional substituent group is a phenyl.

19. The molecular probe according to claim 1, selected from one of the compounds 115.-116.:

| | |
|---|---|
| 115. | 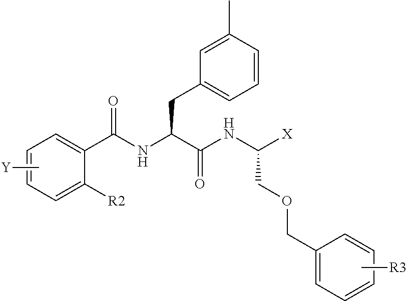 |
| | wherein R2 is a halogen and R3 is COOH or H. |
| 116. | 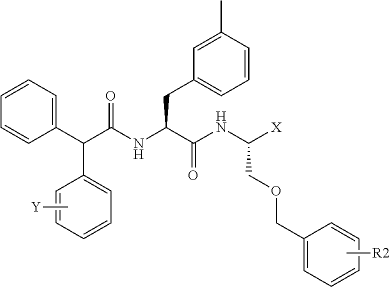 |
| | wherein R2 is COOH or H. |

wherein Y is -L-R1-L1; and R1, L and L1 are as described in claim 1.

20. The molecular probe according to claim 1, selected from one of the compounds 117.-142.:

| | |
|---|---|
| 117. | 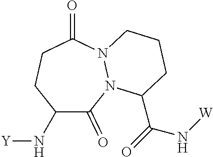 |
| 117a. | 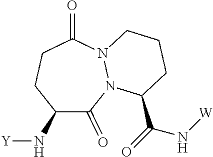 |
| 118. | 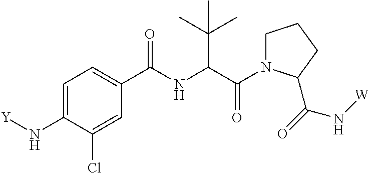 |
| 118a. | 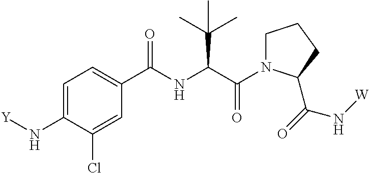 |
| 119. | 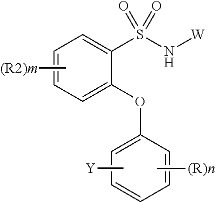 |
| | wherein |
| | R is (C₁-C₅)alkyl, phenyl, (C₅-C₆)cycloalkyl; |
| | R2 is alkyl; and |
| | n and m are independently of each other 0-3. |
| 120. | 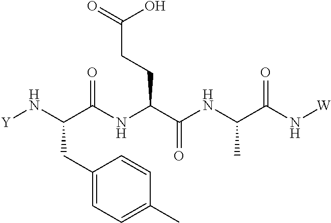 |
| 121. | 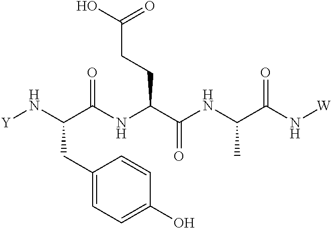 |
| 122. | 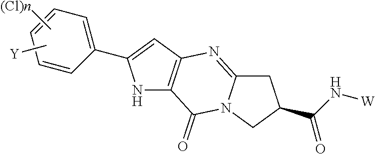 |
| | wherein n is 0 or 1; |
| 123. | 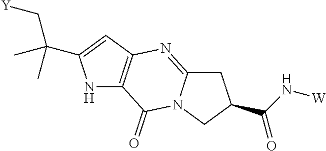 |
| 124. | 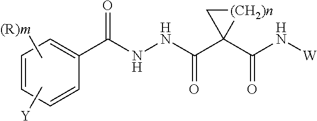 |
| | wherein |
| | n is 1-4; |
| | m is 1 or 2; and |
| | R is methyl or methoxy. |
| 125. | 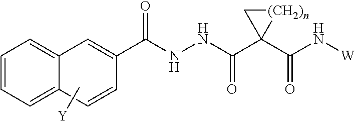 |
| | wherein n is 1-4; |
| 126. | 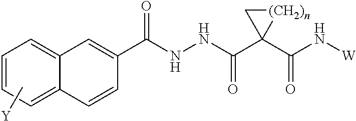 |
| | wherein n is 1-4; |
| 127. | 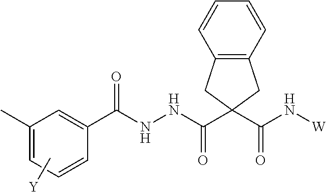 |
| 128. | 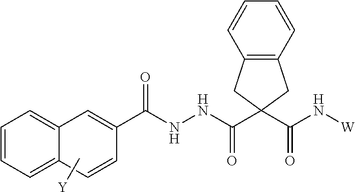 |
| 129. | 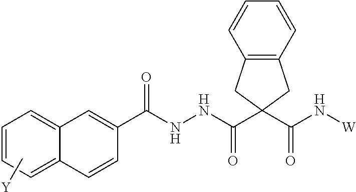 |
| 130. | 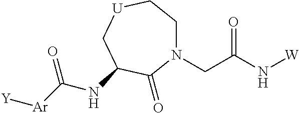 |
| | wherein |
| | U is S or S(O)₂, and |
| | Ar is aryl or heteroaryl; |
| 131. | 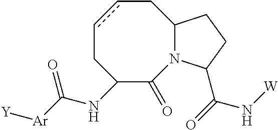 |
| | wherein |
| | Ar is an aryl or heteroaryl group selected from phenyl, |
| | benzothiophene, isoquinolyl, cinnamyl, naphthyl, which is |
| | optionally once or independently twice substituted by |
| | methoxy, chloro, methyl, CF₃, and |
| | wherein |
| | 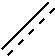 |
| | means either a single or a double bond |
| 132. | 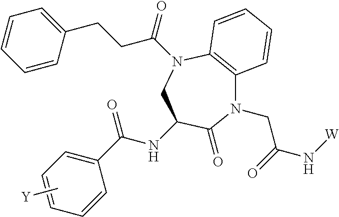 |
| 133. | 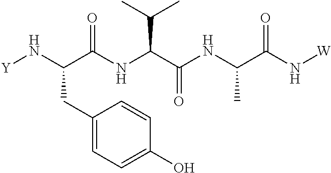 |
| 134. | 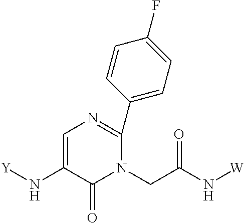 |
| 135 | 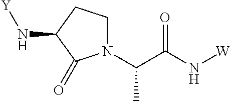 |
| 136. | 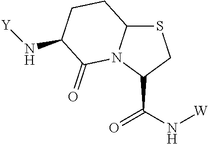 |
| 137. | 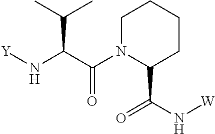 |
| 138. | 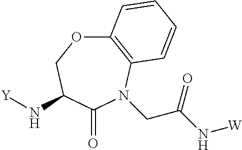 |
| 139. | 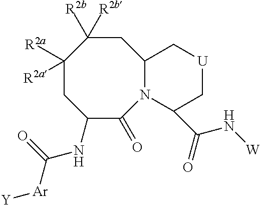 |
| | wherein |
| | Ar is aryl or heteroaryl; |
| | U is CH₂, O or NR9 wherein R9 is hydrogen or (C₁-C₆)alkyl, |
| | aryl, heteroaryl, heterocyclyl; |
| | R^{2a}, R^{2a′}, R^{2b} and R^{2b′} are each independently hydrogen, hydroxyl, |
| | N(R⁶)₂, halogen, (C₁-C₄)alkyl, (C₁-C₄)alkoxy, and mixtures |
| | thereof wherein R⁶ is hydrogen, (C₁-C₆)alkyl, cycloalkyl, |
| | (C₆-C₁₀)aryl; or R^{2a} and R^{2b} can taken together to form a double |
| | bond; |
| 139a. | Compound 139. in the all-(S) configuration; |
| 140. | 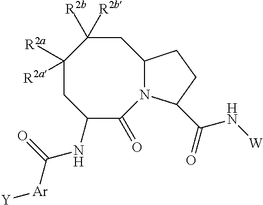 |
| | wherein |
| | Ar is aryl or heteroaryl; |
| | R^{2a}, R^{2a′}, R^{2b} and R^{2b′} are each independently hydrogen, hydroxyl, |
| | N(R⁶)₂, halogen, (C₁-C₄)alkyl, (C₁-C₄)alkoxy, and mixtures |
| | thereof wherein R⁶ is hydrogen, (C₁-C₆)alkyl, cycloalkyl, |
| | (C₆-C₁₀)aryl; or R^{2a} and R^{2b} can taken together to form a double |
| | bond; |
| 140a. | Compound 140. in the all-(S) configuration; |
| 141. | 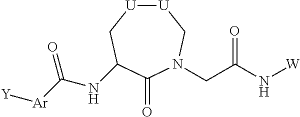 |
| | wherein |
| | Ar is aryl or heteroaryl; and |
| | U is independently selected from: C(R¹)₂; C(O); NR²; S; S(O); |
| | S(O)₂; wherein R¹ and R² are independently hydrogen, |
| | [C(R³)₂]ₚ(CH═CH)_{q}R³, C(═Z)R³, C(═Z)[C(R³)₂]ₚ(CH═CH)_{q}R³, |
| | C(═Z)N(R³)₂, C(═Z)NR³N(R³)₂, CN, CF₃, N(R³)₂, NR³CN, |
| | NR³C(═Z)R³, NRC(═Z)N(R³)₂, NHN(R³)₂, NHOR³, NO₂, OR³, |
| | OCF₃, F, Cl, Br, I, SO₃H, OSO₃H, SO₂N(R³)₂, SO₂R³, |
| | P(O)(OR³)R³, P(O)(OR³)₂; wherein p is 0 to 12; wherein q is 0 to |
| | 12; wherein Z is O, S, NR³; wherein R³ is independently |
| | hydrogen, alkyl, cycloalkyl, aryl, heteroaryl, heterocyclyl; |
| 141a. | Compound 141. in the all-(S) configuration; |
| 142. | 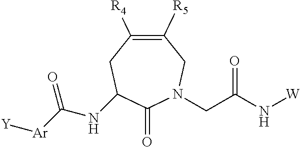 |
| | wherein |
| | Ar is aryl or heteroaryl; and R⁴ and R⁵ are independently selected |
| | from: C(R¹)₂; C(O); NR²; S; S(O); S(O)₂; wherein R¹ and R² are |
| | independently hydrogen, [C(R³)₂]ₚ(CH═CH)_{q}R³, C(═Z)R³, |
| | C(═Z)[C(R³)₂]ₚ(CH═CH)_{q}R³, C(═Z)N(R³)₂, |
| | C(═Z)NR³N(R³)₂, CN, CF₃, N(R³)₂, NR³CN, NR³C(═Z)R³, |
| | NRC(═Z)N(R³)₂, NHN(R³)₂, NHOR³, NO₂, OR³, OCF₃, |
| | F, Cl, Br, I, SO₃H, OSO₃H, SO₂N(R³)₂, SO₂R³, P(O)(OR³)R³, |
| | P(O)(OR³)₂; wherein p is 0 to 12; wherein q is 0 to 12; wherein |
| | Z is O, S, NR³; wherein R³ is independently hydrogen, alkyl, |
| | cycloalkyl, aryl, heteroaryl, heterocyclyl; |
| 142a. | Compound 142. in the all-(S) configuration; |

wherein independently of each other the variables in the compounds 117. to 142a. are defined as indicated in the definition next to the respective compound; Y is -L-R1-L1; and R1, L and L1 are as described in claim 1; and 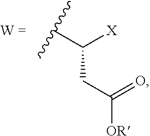
or W═X,
wherein X is a group selected from 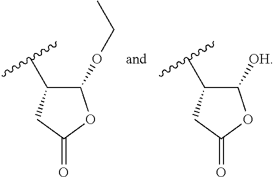
and R′ is H or C₁-C₆-alkyl.

21. The molecular probe according to claim 1, selected from one of the compounds 143.-155.:

| | |
|---|---|
| 143. | 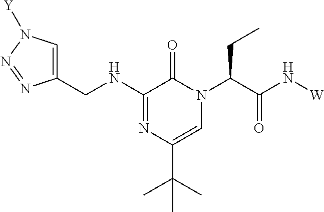 |
| 144. | 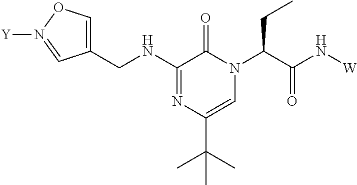 |
| 145. | 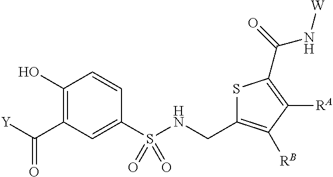 |
| | wherein R^{A} and R^{B} are independently hydrogen, (C₁-C₆)alkyl, |
| | hydroxyl, (C₁-C₆)alkoxy or halogen; |
| 146. | 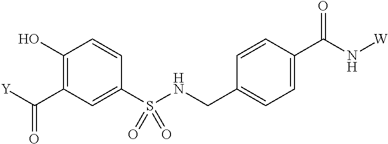 |
| 147. | 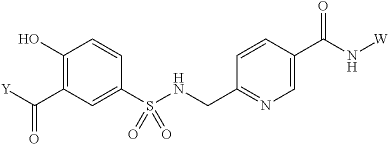 |
| 148. | 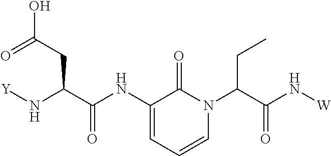 |
| 149. | 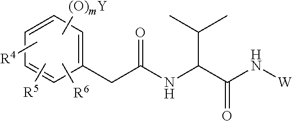 |
| | wherein |
| | m is 0 or 1; and |
| | R⁴, R⁵ and R⁶ are independently selected from the group consisting |
| | of: |
| | 1) H, |
| | 2) halogen, |
| | 3) (C₁-C₄)alkoxy optionally substituted with 1-3 halogen atoms, |
| | 4) NO₂, |
| | 5) OH, |
| | 6) benzyloxy, the benzyl portion of which is optionally substituted |
| | with 1-2 members selected from the group consisting of: halogen, |
| | CN, (C₁-C₄)alkyl and (C₁-C₄)alkoxy, said alkyl and alkoxy being |
| | optionally substituted with 1-3 halogen groups, |
| | 7) NH(C₁-C₄)acyl, |
| | 8) (C₁-C₄)acyl, |
| | 9) O—(C₁-C₄)alkyl-CO₂H, optionally esterified with a (C₁-C₆)alkyl |
| | or a (C₅-C₇)cycloalkyl group, |
| | 10) CH═CH—CO₂H, |
| | 11) CO₂H, |
| | 12) (C₁-C₅)alkyl-CO₂H, |
| | 13) C(O)NH₂, optionally substituted on the nitrogen atom by 1-2 |
| | (C₁-C₄)alkyl groups; |
| | 14) (C₁-C₅)alkyl-C(O)NH₂, optionally substituted on the nitrogen |
| | atom by 1-2 (C₁-C₄)alkyl groups; |
| | 15) S(O)₀₋₂—(C₁-C₄)alkyl; |
| | 16) (C₁-C₂)alkyl-S(O)₀₋₂—(C₁-C₄)alkyl; |
| | 17) S(O)₀₋₂—(C₁-C₆)alkyl or S(O)₀₋₂-phenyl, said alkyl and phenyl |
| | portions thereof being optionally substituted with 1-3 members |
| | selected from the group consisting of: halogen, CN, (C₁-C₄)alkyl |
| | and (C₁-C₄)alkoxy, said alkyl and alkoxy being optionally |
| | substituted by 1-3 halogen groups, |
| | 18) benzoyl optionally substituted by 1-2 members selected |
| | from the group consisting of: halogen, CN, (C₁-C₄)alkyl and |
| | (C₁-C₄)alkoxy, said alkyl and alkoxy groups being optionally |
| | substituted by 1-3 halogen groups, |
| | 19) phenyl or naphthyl, optionally substituted with 1-2 members |
| | selected from the group consisting of: halogen, CN, (C₁-C₄)alkyl |
| | and (C₁-C₄)alkoxy, said alkyl and alkoxy being optionally |
| | substituted with 1-3 halogen groups, |
| | 20) CN, |
| | 21) (C₁-C₄)alkylene-HET2, wherein HET2 represents a 5-7 |
| | membered aromatic or non-aromatic ring containing 1-4 |
| | heteroatoms selected from O, S, NH and N(C₁-C₄) and optionally |
| | containing 1-2 oxo groups, and optionally substituted with 1-3 |
| | (C₁-C₄)alkyl, OH, halogen or (C₁-C₄)acyl groups; |
| | 22) O—(C₁-C₄)alkyl-HET3, wherein HET3 is a 5 or 6 membered |
| | aromatic or non-aromatic ring containing from 1 to 3 heteroatoms |
| | selected from O, S and N, and optionally substituted with one or |
| | two groups selected from halogen and (C₁-C₄)alkyl, and optionally |
| | containing 1-2 oxo groups, and |
| | 23) HET4, wherein HET4 is a 5 or 6 membered aromatic or non- |
| | aromatic ring, and the benzofused analogs thereof, containing |
| | from 1 to 4 heteroatoms selected from O, S and N, and is |
| | optionally substituted by one or two groups selected from halogen, |
| | (C₁-C₄)alkyl and (C₁-C₄)acyl; and wherein halogen includes F, Cl, |
| | Br and I; |
| 149a. | Compound 149. in the all-(S) configuration; |
| 150. | 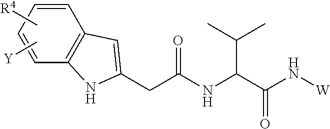 |
| | wherein R⁴ is selected from the group consisting of: |
| | 1) H, |
| | 2) halogen, |
| | 3) (C₁-C₄)alkoxy optionally substituted with 1-3 halogen atoms, |
| | 4) NO₂, |
| | 5) OH, |
| | 6) benzyloxy, the benzyl portion of which is optionally substituted |
| | with 1-2 members selected from the group consisting of: halogen, |
| | CN, (C₁-C₄)alkyl and (C₁-C₄)alkoxy, said alkyl and alkoxy being |
| | optionally substituted with 1-3 halogen groups, |
| | 7) NH(C₁-C₄)acyl, |
| | 8) (C₁-C₄)acyl, |
| | 9) O—(C₁-C₄)alky1-CO₂H, optionally esterified with a |
| | (C₁-C₆)alkyl or a (C₅-C₇)cycloalkyl group, |
| | 10) CH═CH—CO₂H, |
| | 11) CO₂H, |
| | 12) (C₁-C₅)alkyl-CO₂H, |
| | 13) C(O)NH₂, optionally substituted on the nitrogen atom by 1-2 |
| | (C₁-C₄)alkyl groups; |
| | 14) (C₁-C₅)alkyl-C(O)NH₂, optionally substituted on the nitrogen |
| | atom by 1-2 (C₁-C₄)alkyl groups; |
| | 15) S(O)₀₋₂—(C₁-C₄)alkyl; |
| | 16) (C₁-C₂)alkyl-S(O)₀₋₂—(C₁-C₄)alkyl; |
| | 17) S(O)₀₋₂—(C₁-C₆)alkyl or S(O)₀₋₂-phenyl, said alkyl and phenyl |
| | portions thereof being optionally substituted with 1-3 members |
| | selected from the group consisting of: halogen, CN, (C₁-C₄)alkyl |
| | and (C₁-C₄)alkoxy, said alkyl and alkoxy being optionally |
| | substituted by 1-3 halogen groups, |
| | 18) benzoyl optionally substituted by 1-2 members selected from |
| | the group consisting of: halogen, CN, (C₁-C₄)alkyl and |
| | (C₁-C₄)alkoxy, said alkyl and alkoxy groups being optionally |
| | substituted by 1-3 halogen groups, |
| | 19) phenyl or naphthyl, optionally substituted with 1-2 members |
| | selected from the group consisting of: halogen, CN, (C₁-C₄)alkyl |
| | and (C₁-C₄)alkoxy, said alkyl and alkoxy being optionally |
| | substituted with 1-3 halogen groups, |
| | 20) CN, |
| | 21) (C₁-C₄)alkylene-HET2, wherein HET2 represents a 5-7 |
| | membered aromatic or non-aromatic ring containing 1-4 |
| | heteroatoms selected from O, S, NH and N(C₁-C₄) and optionally |
| | containing 1-2 oxo groups,and optionally substituted with 1-3 |
| | (C₁-C₄)alkyl, OH, halogen or (C₁-C₄)acyl groups; |
| | 22) O—(C₁-C₄)alkyl-HET3, wherein HET3 is a 5 or 6 membered |
| | aromatic or non-aromatic ring containing from 1 to 3 heteroatoms |
| | selected from O, S and N, and optionally substituted with one or |
| | two groups selected from halogen and (C₁-C₄)alkyl, and optionally |
| | containing 1-2 oxo groups, and |
| | 23) HET4, wherein HET4 is a 5 or 6 membered aromatic or non- |
| | aromatic ring, and the benzofused analogs thereof, containing from |
| | 1 to 4 heteroatoms selected from O, S and N, and is optionally |
| | substituted by one or two groups selected from halogen, |
| | (C₁-C₄)alkyl and (C₁-C₄)acyl; and wherein halogen includes F, Cl, |
| | Br and I; |
| 150a. | Compound 150. in the all-(S) configuration; |
| 151. | 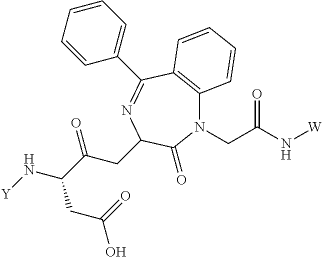 |
| 152. | 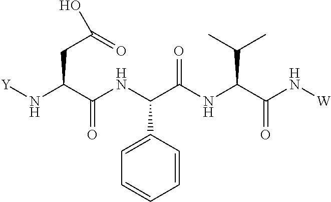 |
| 153. | 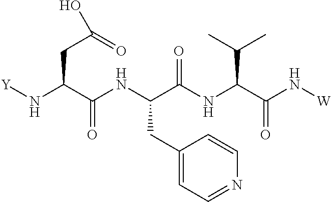 |
| 154. | 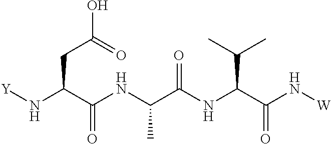 |
| 155. | 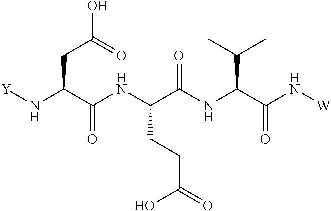 |

wherein independently of each other the variables in the compounds 143. to 155. are defined as indicated in the definition next to the respective compound; Y is -L-R1-L1; and R1, L and L1 are as described in claim 1; and 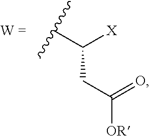
or W═X,
wherein X is a group selected from 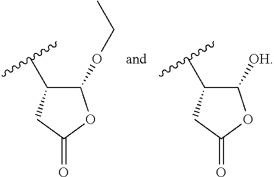
and R′ is H or C₁-C₆-alkyl.

22. The molecular probe according to claim 1, selected from one of the compounds 156.-157.:

| | |
|---|---|
| 156. | 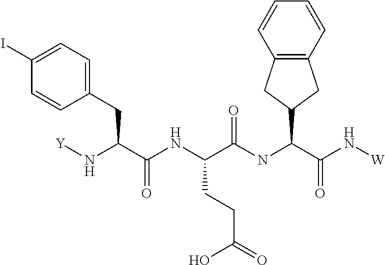 |
| 157. | 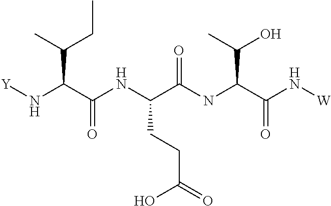 |

wherein independently of each other the variables in the compounds 156. to 157. are defined as indicated in the definition next to the respective compound; Y is -L-R1-L1; and R1, L and L1 are as described in claim 1; and 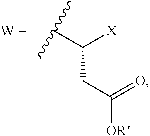
or W═X,
wherein X is a group selected from 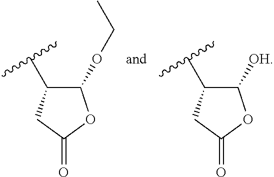
and R′ is H or C₁-C₆-alkyl.

23. The molecular probe according to claim 1, selected from a compound of the formula 158.:

| | |
|---|---|
| 158. | 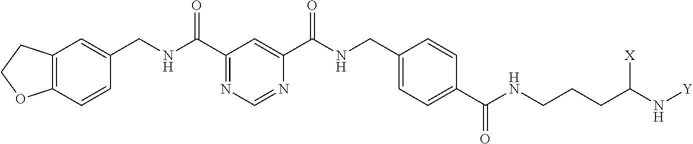 |

wherein Y is -L-R1-L1; and R1, L and L1 are as described in claim 1 and X is a hydrogen.

24. The molecular probe according to claim 1, selected from one of the compounds 159.-167.:

| | |
|---|---|
| 159. | 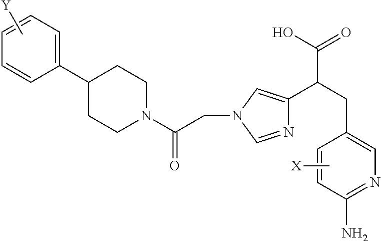 |
| 160. | 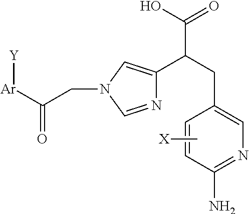 |
| | wherein Ar is aryl; |
| 161. | 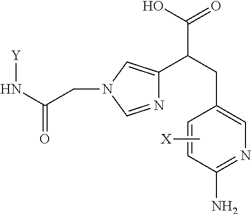 |
| 162. | 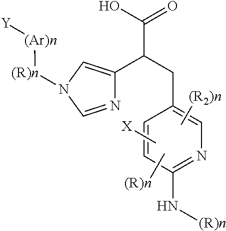 |
| | wherein R is alkyl, cycloalkyl; R₂ is halogen; Ar is aryl; and each n |
| | is independently 0 or 1; |
| 163. | 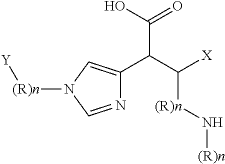 |
| | wherein R is alkyl, cycloalkyl; and each n is independently 0 or 1; |
| 164. | 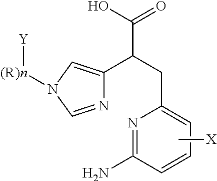 |
| | wherein R is alkyl, cycloalkyl; and n is 0 or 1; |
| 165. | 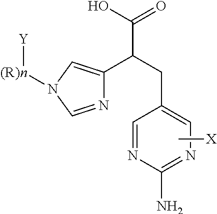 |
| | wherein R is alkyl, cycloalkyl; and n is 0 or 1 |
| 166. | 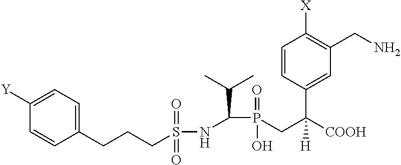 |
| 167. | 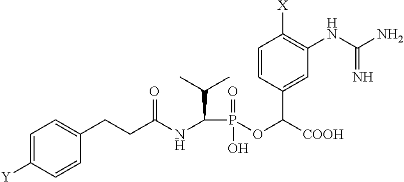 |

wherein Y is -L-R1-L1; and R1, L and L1 are as described in claim 1, and X is a hydrogen atom.

25. The molecular probe according to any one of claims 17 to 24, wherein
L is a direct bond or a group selected from 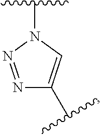
—(NRx)-, —O—, —C═N—, —C(═O)—, —C(═O)—NH—, —NH—C(═O)—, —C(═O)H, —CRx=CRy-, —C≡C— and phenyl, wherein Rx and Ry are independently H or (C₁-C₆)alkyl;
R1 is a straight or branched chain alkylene group with 1 to 300 carbon atoms, wherein optionally
(a) one or more carbon atoms are replaced by oxygen;
(b) one or more carbon atoms are replaced by nitrogen carrying a hydrogen atom, and the adjacent carbon atoms are substituted by oxo, representing an amide function —NH—CO—;
(c) one or more carbon atoms are replaced by an ester function —O—CO—;
(d) the bond between two adjacent carbon atoms is a double or a triple bond; or
(e) two adjacent carbon atoms are replaced by a disulfide linkage.
or a combination thereof;
L1 is biotin or methotrexate or a fluorophore selected from the group consisting of a dimethylaminocoumarin derivative, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647; and
X is a group selected from 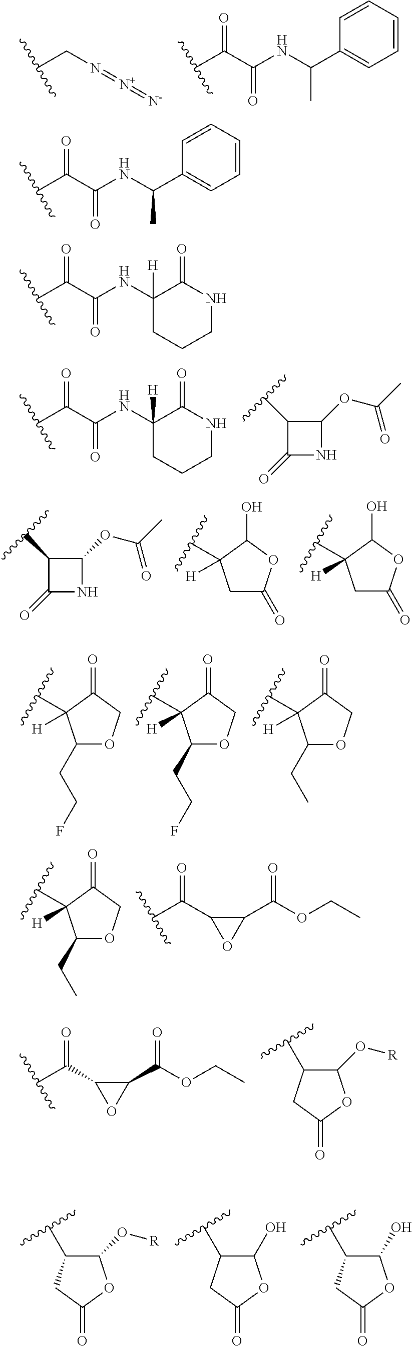
wherein R is alkyl or aryl.

26. The molecular probe according to claim 25, wherein
L is a group selected from 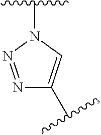
—(NRx)-, —O—, —C═N—, —C(═O)—, —C(═O)—NH—, —NH—C(═O)—, —C(═O)H, —CRx=CRy-, —C≡C— and phenyl, wherein Rx and Ry are independently H or (C₁-C₆)alkyl, or a direct bond;
R1 is alkyl or a straight or branched chain alkylene group with 1 to 50 carbon atoms, wherein one or more carbon atoms are replaced by oxygen;
L1 is a fluorophore selected from the group consisting of a dimethylaminocoumarin derivative, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647; and
X is a nitrile group or a group selected from

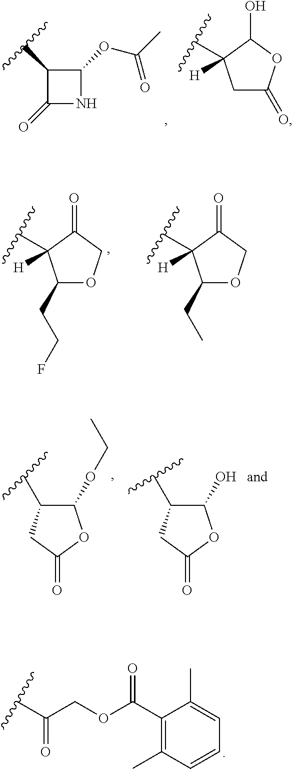

27. A method for molecular imaging in vitro, in cell-culture experiments, ex-vivo experiments or in a living organism, the method comprising the use of a probe of the formula (I) according to claim 1.
